(19) United States
(12) Patent Application Publication
(10) Pub. No.: US 2025/0354995 A1
(43) Pub. Date: Nov. 20, 2025

(54) MAMMALIAN DISPLAY PLATFORM FOR MULTISPECIFIC ANTIGEN BINDING PROTEINS

(71) Applicant: Immatics Biotechnologies GmbH, Tuebingen (DE)

(72) Inventors: Janine DILCHERT, Schaafheim (DE); Sebastian BUNK, Tübingen (DE); Martin HOFMANN, Tübingen (DE); Felix UNVERDORBEN, Tübingen (DE); Gabriele PSZOLLA, Tübingen (DE)

(21) Appl. No.: 18/860,347

(22) PCT Filed: Apr. 27, 2023
(86) PCT No.: PCT/EP2023/061180

§ 371 Date: Oct. 25, 2024

Related U.S. Application Data
(60) Provisional application No. 63/336,881, filed on Apr. 29, 2022.

(30) Foreign Application Priority Data
Apr. 29, 2022 (EP) 22171038.7

(51) Int. Cl. G01N 33/68; C12N 15/10; G01N 33/50

(52) U.S. Cl. CPC G01N 33/6845; C12N 15/1037; G01N 33/5011

(57) ABSTRACT

The present invention relates to a method of providing a multispecific antigen binding protein (ABP), wherein the multispecific ABP is comprising at least one T-cell receptor (TCR)-derived binding site.

### CROSS-REFERENCE TO RELATED APPLICATIONS

This application is a National Stage entry of International Application No. PCT/EP2023/061180, filed 27 Apr. 2023, which claims priority to European Patent Application No. 22171038.7, filed 29 Apr. 2022, and U.S. Provisional Application No. 63/336,881, filed 29 Apr. 2022.

### REFERENCE TO SEQUENCE LISTING SUBMITTED AS AN XML FILE (.xml)

A Sequence Listing is submitted herewith as an XML file named “3000058-024001 Substitute SL” created on 10 Jul. 2025 and having a size of 183,334 bytes as permitted under 37 C.F.R. § 1.821 (c). The material in the aforementioned file is hereby incorporated by reference in its entirety.

### BACKGROUND

### Field

The present invention relates to a method of providing a multispecific antigen binding protein (ABP), wherein the multispecific ABP is comprising at least one T-cell receptor (TCR)-derived binding site.

For the immunotherapeutic treatment of cancer bispecific antibodies have been developed that retarget T cells to cancer cells to induce T cell activation and tumor cell lysis (Labrijn et al Nat Rev Drug Discov, 2019; 18:585-608). Although this concept of bispecific T cell engagers was already proposed in the mid-1980s, it took more than 20 years until market approval of the first T cell retargeting bispecific molecules, catumaxomab (EpCAM×CD3), was approved 2009 in the European Union and blinatumomab (CD19×CD3) was approved five years later. Notably, the first T cell receptor (TCR)-based bispecific molecule, a gp100×CD3 ImmTAC molecule, was recently approved for the treatment of metastatic uveal melanoma (Mullard, A; Nat Rev Drug Discov 2022). Many clinical trials investigating bispecific antibody therapies were initiated since the first approval, but still a large number of patients still do not benefit from these efforts. This is often either related to the lack of effective therapeutics or the lack of selective cancer targets (Sambi, M et al; J Oncol 2019; 2019:4508794; Ventola C L; J Formulary Management 2017; 42:514-21).

TCR-based therapeutics have the potential to overcome the shortcomings of antibody-based therapeutics, which naturally bind only cancer antigens expressed on the cell surface. TCR-based therapeutics can in addition target the very relevant proportion of cancer antigens expressed only intracellularly. TCRs bind to proteolyzed intra- and extracellular peptides presented in the context of the human leukocyte antigen complex (HLA). Their therapeutic potential can be exploited mainly in two different ways, (a) adoptive T cell therapies and (b) soluble bispecific TCR molecules. The generation of multispecific TCR containing molecules requires a complex affinity maturation to increase the low affinity of TCRs (1-100 μM) by several orders of magnitude. Furthermore, maturation may also be required to increase solubility and stability of the TCRs to prevent potential issues related to insolubility and high aggregation propensity (Lowe K L et al., Cancer Treat Rev 2019; 77:35-43; Vafa O et al., Frontiers Oncol 2020; 10:446; Matsui K et al., Science 1991; 254:1788-91). Phage and yeast display systems have been successfully used for TCR maturation. However, their inability to adequately reflect human post-translational modifications, such as glycosylation, can be of disadvantage for maturation of TCRs constituting glycoproteins by nature. This aspect might be even more relevant if later production of the TCR-based bispecifics utilizes mammalian expression hosts (Birch J R et al., Adv Drug Deliver Rev 2006; 58:671-85; Wurm F M, Nat Biotechnol 2004; 22:1393-8).

Thus there is a need for a reliable system allowing the maturation of multispecific TCR containing binders in mammalian cells.

### SUMMARY OF THE INVENTION

An improved maturation process of multispecific TCR containing therapeutics is disclosed herein utilizing a maturation platform based on mammalian cells allowing TCR maturation in the context of a final multispecific TCR format.

In a first aspect, the present invention provides a method of providing a multispecific antigen binding protein (ABP), wherein the multispecific ABP is comprising at least one T-cell receptor (TCR)-derived binding site, comprising the steps of:
(I) generating a library of nucleic acids encoding multispecific ABPs, which comprises different variants of the multispecific ABPs;
(II) providing a multitude of mammalian cells each expressing one member of the library of step (I) on their cell surface;
(III) contacting the mammalian cells with a first target of the multispecific ABP and selecting a mammalian cell expressing a multispecific ABP binding to the first target;
(IV) optionally contacting the mammalian cell selected in step (III) with a further target of the multispecific ABP and selecting a mammalian cell expressing a multispecific ABP binding to the further target;
(V) isolating a multispecific ABP from the selected mammalian cell of step (III) or if performed step (IV).

### LIST OF FIGURES

In the following, the content of the figures comprised in this specification is described. In this context please also refer to the detailed description of the invention above and/or below.

FIG. 1: Vector design and process overview for generation of landing pad-containing CHO cell line refers to A) pJD1_GFP vector for stable GFP integration into CHO genome. The hCMV promotor driven GFP expression cassette is flanked by a 5′ FRT site and a 3′ FRT F3 site followed by a SV40 promotor driven neomycin resistance cassette. B) pJD1_RFP donor vector for RMCE. The hCMV promotor driven RFP expression cassette is flanked by a 5′ FRT site and a 3′ FRT F3 site followed by a SV40 promotor driven neomycin resistance cassette. C) RMCE process for generation of a landing pad-containing CHO cell line. Upon integration of the GFP-expressing landing pad into the CHO genome a RMCE was performed to exchange with an RFP expressing cassette. CHO cells showing only RFP expression were single cell sorted and a stable RFP expressing clone was expanded as the landing pad containing cell line.

FIG. 2: Flow cytometric analysis of CHO cells during landing pad integration refers to A) Gating strategy of the enrichment of high expressing GFP cells. The top 2% of the GFP expressing cells were sorted two times. B) The histogram plot indicates the GFP expression levels in comparison to non-transfected CHO cells (negative control) C) GFP and RFP expression of CHO cells subjected to RMCE with pJD1_RFP donor vector and RNA-encoded Flp recombinase. RFP-positive/GFP-negative cells were sorted and single clones were generated for analysis of the integration site.

FIG. 3: Stability of RFP expression of CHO clone RFP_A03 selected for landing pad containing CHO cell line refers to A) RFP MFI values and B) percentage of RFP-positive cells of RFP_A03 CHO cells during long-term culture of 135 days.

FIG. 4: Vector design and overview of RMCE-mediated process for TCER library generation in CHO cells refers to A) pJD1_TCER donor vector with hCMV promotor-driven expression cassettes for TCER chain 1 and 2, which are flanked by a 5′ FRT and a 3′ FRT F3 site. B) Molecular structure of a TCER molecule. TCER chain 1 encodes for the variable TCR alpha chain (Va.), the variable light chain (VL) of an UCHT1 anti-CD3 antibody followed by IgG1 constant domains CH2 and CH3 modified with specific mutations to ablate Fc gamma receptor binding and complement activation as well as knob-forming mutations. TCER chain 2 encodes for the variable heavy chain (VH) of the anti-CD3 antibody and the variable TCR beta chain (Vβ) followed by IgG1 constant domains CH2 and CH3 modified with specific mutations to ablate Fc gamma receptor binding and complement activation as well as hole-forming mutations. C) RMCE process for TCER library generation in CHO cells. The RFP locus of the RFP_A03 CHO cells was exchanged via RCME with TCER containing donor vectors encoding for all potential combinations of CDR variants (see also Table 2) of a PRAME-specific model TCR.

FIG. 5: Flow-cytometric analysis of PRAME and similar peptide binding of CHO-displayed TCER candidates refers to A) Target binding was analyzed with PRAME pHLA monomer applied at concentrations ranging from 100 nM to 10 pM. B) Binding of TCER candidates to 10 nM similar peptide tetramers comprising each of 11 different similar peptides (Table 1) in relation to 10 nM PRAME peptide tetramer. Each datapoint represents the mean of triplicate measurements with the respective standard error of mean (SEM).

FIG. 6: Assessment of tumor cell lysis mediated by the selected TCER candidates refers to PBMC of a healthy human donor were co-cultured with tumor cells at an effector to target ratio of 10:1 and in presence of increasing TCER concentrations. Lysis of PRAME pHLA positive target cells UACC-257 (1100 cpc), HS695T (400-550 cpc), and A375 (50 cpc) was determined based on LDH-release assay. For control, SET 2 cells were used as a target negative cell line. Each datapoint represents the mean of triplicate measurements with the respective SEM.

FIG. 7: Assessment of TCER-mediated cytokine release from PBMC in response to tumor cells refers to PBMCs of a healthy human donor were co-cultured with tumor cells at an effector to target ratio of 10:1 and in presence of increasing TCER concentrations. CD3- and CD28-binding antibodies were used as positive control. Cytokine levels were determined using the MACSPlex cytotoxic T/NK cell kit. UACC-257 (1100 cpc) was used as PRAME target pHLA positive cell line and SET-2 was used target negative control cell line. Each datapoint represents the mean of a triplicate with the respective SEM.

### DETAILED DESCRIPTIONS OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “about” when used in connection with a numerical value is meant to encompass numerical values within a range having a lower limit that is 5% smaller than the indicated numerical value and having an upper limit that is 5% larger than the indicated numerical value.

The term “antigen binding protein (ABP)” herein refers to polypeptides or binding proteins that are able to specifically bind to at least one antigen. Preferred examples of an ABP are T-cell receptors (TCRs) and antibodies. A variant of an ABP maintains the binding specificity for a certain antigen of the parent ABP. A multispecific ABP has multiple binding valences and binding specificities. A preferred example of a multispecific ABP is a bispecific ABP that has at least two valences and binding specificities for at least two different antigens. A preferred bispecific ABP has two valences and two binding specificities.

The term “binding valence” as used herein refers to the number of antigen binding sites on a single antigen binding protein. For example a monovalent binding involves only a single antigen binding site.

The term “antigen” as used herein refers to a molecule or a portion of a molecule or complex that is capable of being bound by at least one antigen binding site, wherein said one antigen binding site is, for example an antibody derived binding site or a TCR derived binding site.

The term “binding” according to the invention preferably relates to a specific binding.

The term “binding affinity” generally refers to the strength of the sum total of noncovalent interactions between a single binding site of an ABP (e.g., a TCR derived or an antibody derived binding site) and its binding partner (e.g., target or antigen). Unless indicated otherwise, as used herein, “binding affinity” refers to intrinsic binding affinity which reflects a 1:1 interaction between members of a binding pair (e.g. a TCR derived or an antibody derived binding site and its antigen). The affinity of a molecule X for its partner Y can generally be represented by the dissociation constant (Kd). A “higher binding affinity” is represented by a lower Kd value and vice versa. “Specific binding” means that an antigen binding protein binds stronger to a target for which it is specific compared to the binding to another target. An ABP binds stronger to a first target compared to a second target if it binds to the first target with a dissociation constant (Kd) which is lower than the dissociation constant for the second target. The dissociation constant (Kd) for the target to which the ABP binds specifically is more than 10-fold, preferably more than 20-fold, more preferably more than 50-fold, even more preferably more than 100-fold, 200-fold, 500-fold or 1000-fold lower than the dissociation constant (Kd) for the target to which the ABP does not bind specifically.

Accordingly, the term “Kd” (measured in “mol/L”, sometimes abbreviated as “M”) is intended to refer to the dissociation equilibrium constant of the particular interaction between an ABP and a target molecule. Affinity can be measured by common methods known in the art, including but not limited to surface plasmon resonance based assay (such as the BIAcore assay); quartz crystal microbalance assays (such as Attana assay); enzyme-linked immunoabsorbent assay (ELISA); and competition assays (e.g. RIA's). Low-affinity ABPs generally bind antigen slowly and tend to dissociate readily, whereas high-affinity ABPs generally bind antigen faster and tend to remain bound longer. A variety of methods of measuring binding affinity are known in the art, any of which can be used for purposes of the present invention. One example of a method to determine binding affinity is surface plasmon resonance or biolayer interferometry.

Typically, ABPs according to the invention bind with a sufficient binding affinity to their target, for example, with a Kd value of between 100 nM and 1 pM, i.e. 100 nM, 50 nM, 10 nM, 5 nM, 1 nM, 900 pM, 800 pM, 700 pM, 600 pM, 500 pM, 400 pM, 300 pM, 200 pM, 100 pM, 50 pM, 1 pM.

A “T-cell receptor (TCR)” in the context of the present invention is a heterodimeric cell surface protein of the immunoglobulin super-family, which is associated with invariant proteins of the CD3 complex involved in mediating signal transduction. TCRs exist in αβ and γδ forms, which are structurally similar but have quite distinct anatomical locations and probably functions. The extracellular portion of native heterodimeric αβ TCR and γδ TCR each contain two polypeptides, each of which has a membrane-proximal constant domain, and a membrane-distal variable domain. Each of the constant and variable domains include an intra-chain disulfide bond. The variable domains contain the highly polymorphic loops analogous to the complementarity determining regions (CDRs) of antibodies.

The term “TCR” herein denotes TCRs and fragments thereof, as well as single chain TCRs and fragments thereof, in particular variable alpha and beta domains of single domain TCRs, and chimeric, humanized, bispecific or multispecific TCRs.

“Fragments of a TCR” comprise a portion of an intact or native TCR, in particular the antigen binding region or variable region of the intact or native TCR. Examples of TCR fragments include fragments of the α, β, δ, γ chain, such as Vα-Ca or Vβ-Cβ or portions thereof, such fragments might also further comprise the corresponding hinge region or single variable domains, such as Vα, Vβ, Vδ, Vγ, single chain VαVβ fragments or bispecific and multispecific TCRs formed from TCR fragments. Fragments of a TCR exert identical functions compared to the naturally occurring full-length TCR, i.e. fragments selectively and specifically bind to their target peptide.

“Native” as used for example in the wording “native TCR” refers to a wildtype TCR. Native alpha-beta heterodimeric TCRs have an alpha chain and a beta chain. Each alpha chain comprises variable, joining and constant regions, and the beta chain also usually contains a short diversity region between the variable and joining regions, but this diversity region is often considered as part of the joining region. The constant, or C, regions of TCR alpha and beta chains are referred to as TRAC and TRBC respectively (Lefranc, (2001), Curr Protoc Immunol Appendix 1: Appendix 10). Each variable region, herein referred to as alpha variable domain and beta variable domain, comprises three Complementarity Determining Regions (CDRs) embedded in a framework sequence, one being the hypervariable region named CDR3. The alpha variable domain CDRs are herein referred to as CDRa1, CDRa2, CDRa3, and the beta variable domain CDRs are herein referred to as CDRb1, CDRb2, CDRb3. There are several types of alpha chain variable (Valpha) regions and several types of beta chain variable (Vbeta) regions distinguished by their framework, CDR1 and CDR2 sequences, and by a partly defined CDR3 sequence. The Valpha types are referred to in IMGT nomenclature by a unique TRAV number, Vbeta types are referred in IMGT nomenclature to by a unique TRBV number (Folch and Lefranc, (2000), Exp Clin Immunogenet 17(1): 42-54; Scaviner and Lefranc, (2000), Exp Clin Immunogenet 17(2): 83-96; LeFranc and LeFranc, (2001), “T cell Receptor Factsbook”, Academic Press). For more information on immunoglobulin antibody and TCR genes see the international ImMunoGeneTics information system*, Lefranc M-P et al (Nucleic Acids Res. 2015 January; 43 (Database issue):D413-22; and http://www.imgt.org/). A conventional TCR antigen-binding site, therefore, includes, usually, six CDRs, comprising the CDR set from each of an alpha and a beta chain variable region, wherein CDR1 and CDR3 sequences are relevant to the recognition and binding of the peptide antigen that is bound to the HLA protein and the CDR2 sequences are relevant to the recognition and binding of the HLA protein.

Analogous to antibodies, “TCR Framework Regions” (FRs) refer to amino acid sequences interposed between CDRs, i.e. to those portions of TCR alpha and beta chain variable regions that are to some extent conserved among different TCRs in a single species. The alpha and beta chains of a TCR each have four FRs, herein designated FR1-a, FR2-a, FR3-a, FR4-a, and FR1-b, FR2-b, FR3-b, FR4-b, respectively. Accordingly, the alpha chain variable domain may thus be designated as (FR1-a)-(CDRa1)-(FR2-a)-(CDRa2)-(FR3-a)-(CDRa3)-(FR4-a) and the beta chain variable domain may thus be designated as (FR1-b)-(CDRb1)-(FR2-b)-(CDRb2)-(FR3-b)-(CDRb3)-(FR4-b).

In the context of the invention, CDR/FR definition in an α or β chain or α γ or δ chain is to be determined based on IMGT definition (Lefranc et al. Dev. Comp. Immunol., 2003, 27(1):55-77; www.imgt.org). Accordingly, CDR/FR amino acid positions when related to TCR or TCR derived domains are indicated according to said IMGT definition.

With respect to gamma/delta TCRs, the term “TCRgamma variable domain” as used herein refers to the concatenation of the TCR gamma V (TRGV) without leader region (L), and TCR gamma J (TRGJ) regions; and the term TCR gamma constant domain refers to the extracellular TRGC region, or to a C-terminal truncated TRGC sequence. Likewise the term “TCR delta variable domain” refers to the concatenation of the TCR delta V (TRDV) without leader region (L), and TCR delta D/J (TRDD/TRDJ) regions; and the term TCR delta constant domain refers to the extracellular region, or to a C-terminal truncated TRDC sequence.

The “major histocompatibility complex” (MHC) in the context of the present invention is a set of cell surface proteins essential for the acquired immune system to recognize foreign molecules in vertebrates, which in turn determines histocompatibility. The main function of MHC molecules is to bind to antigens derived from pathogens and display them on the cell surface for recognition by the appropriate T cells. The human MHC is also called the HLA (human leukocyte antigen) complex (often just the HLA). The MHC gene family is divided into three subgroups: class I, class II, and class III. Complexes of peptide and MHC class I are recognized by CD8-positive T cells bearing the appropriate T cell receptor (TCR), whereas complexes of peptide and MHC class II molecules are recognized by CD4-positive-helper-T cells bearing the appropriate TCR. Since both types of response, CD8 and CD4 dependent, contribute jointly and synergistically to the anti-tumor effect, the identification and characterization of tumor-associated antigens and corresponding T cell receptors is important in the development of cancer immunotherapies such as vaccines and cell therapies. The HLA-A gene is located on the short arm of chromosome 6 and encodes the larger, α-chain, constituent of HLA-A. Variation of HLA-A α-chain is key to HLA function. This variation promotes genetic diversity in the population. Since each HLA has a different affinity for peptides of certain structures, greater variety of HLAs means greater variety of antigens to be ‘presented’ on the cell surface. Each individual can express up to two types of HLA-A, one from each of their parents. Some individuals will inherit the same HLA-A from both parents, decreasing their individual HLA diversity; however, the majority of individuals will receive two different copies of HLA-A. This same pattern follows for all HLA groups. In other words, every single person can only express either one or two of the 2432 known HLA-A alleles.

The MHC class I HLA protein in the context of the present invention may be an HLA-A, HLA-B or HLA-C protein, preferably HLA-A protein, more preferably HLA-A*02.

“HLA-A*02” signifies a specific HLA allele, wherein the letter A signifies the gene and the suffix “*02” indicates the A2 serotype.

In the MHC class I dependent immune reaction, peptides not only have to be able to bind to certain MHC class I molecules expressed by tumor cells, they subsequently also have to be recognized by T cells bearing specific T cell receptors (TCR).

A “MHC-associated peptide epitope” in the context of the present invention is thus an epitope on a peptide that is presented by a MHC molecule and that can be bound by an ABP (in particular a TCR). Preferably a MHC class I associated peptide has a length of 8 to 11 amino acids, preferably 9 to 10, most preferably 9 amino acids. Preferably a MHC class II associated peptide has a length of 13 to 25 amino acids.

In an “antibody” also called immunoglobulin two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site (synonym to antibody binding site) and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

In the context of the invention, the antibody or immunoglobulin is an IgM, IgD, IgG, IgA or IgE, preferably IgG.

“Antibody Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. Accordingly, the light chain variable domain may thus be designated as (FR1-L)-(CDR1-L)-(FR2-L)-(CDR2-L)-(FR3-L)-(CDR3-L)-(FR4-L) and the heavy chain variable domain may thus be designated as (FR1-H)-(CDR1-H)-(FR2-H)-(CDR2-H)-(FR3-H)-(CDR3-H)-(FR4-H).

In the context of the invention, CDR/FR definition in an immunoglobulin light or heavy chain is to be determined based on Kabat numbering (Kabat E A, Te, Wu T, Foeller C, Perry H M, Gottesman K S. (1992) Sequences of Proteins of Immunological Interest.).

The term “antibody” denotes antibodies and fragments thereof, as well as single domain antibodies and fragments thereof, in particular a variable heavy chain of a single domain antibody, and chimeric, humanized, bispecific or multispecific antibodies.

A “conventional antibody” as herein referred to is an antibody that has the same domains as an antibody isolated from nature and comprises antibody derived CDRs and Framework regions. In analogy, a “conventional TCR” as herein referred to is a TCR that comprises the same domains as a native TCR and TCR derived CDRs and Framework regions.

Amino acid residues that are part of a CDR (in TCR derived and antibody derived CDRs) will typically not be altered, although in certain cases it may be desirable to alter individual CDR amino acid residues, for example to remove a glycosylation site, a deamidation site, an isomerization site, or an undesired cysteine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, in particular byway of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in a CDR sequence, it may therefore be desirable to remove the site, typically by conservative substitution to remove one of the implicated residues. Substitution in a CDR sequence to remove one of the implicated residues is also intended to be encompassed by the present invention.

“Fragments of antibodies” in the context of the present invention comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments. A fragment of an antibody may also be a single domain antibody, such as a heavy chain antibody or VHH.

The term “Fab” denotes an antibody fragment having a molecular weight of about 50,000 Dalton and antigen binding activity, in which about a half of the N-terminal side of H chain and the entire L chain, among fragments obtained by treating IgG with a protease, e.g. papain, are bound together through a disulfide bond.

The term “Fc domain” as used in the context of the present invention encompasses native Fc domains and Fc domain variants and sequences as further defined herein below. As with Fc variants and native Fc molecules, the term “Fc domain” includes molecules in monomeric or multimeric form, whether digested from whole antibody or produced by other means.

The term “native Fc” as used herein refers to a molecule comprising the sequence of a non-antigen-binding fragment resulting from digestion of an antibody or produced by other means, whether in monomeric or multimeric form, and may contain the hinge region. The original immunoglobulin source of the native Fc is, in particular, of human origin and can be any of the immunoglobulins, preferably IgG1 or IgG2, most preferably IgG1. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a native Fc is a disulfide-bonded dimer resulting from papain digestion of an IgG. The term “native Fc” as used herein is generic to the monomeric, dimeric, and multimeric forms. One example of a native Fc amino acid sequence is the amino acid sequence of SEQ ID NO: 141, which is the native Fc amino acid sequence of IGHG1*01.

The “hinge” or “hinge region” or “hinge domain” refers typically to the flexible portion of a heavy chain located between the CH1 domain and the CH2 domain. It is approximately 25 amino acids long, and is divided into an “upper hinge”, a “middle hinge” or “core hinge” and a “lower hinge”. A “hinge subdomain” refers to the upper hinge, middle (or core) hinge or the lower hinge. The amino acids sequences of the hinges of an IgG1, IgG2, IgG3 and IgG4 molecule are indicated herein below:

IgG1:
(SEQ ID No. 001)
E216PKSCDKTHTCPPCPAPELLG
IgG2:
(SEQ ID No. 002)
E216RKCCVECPPCPAPPVAGP
IgG3:
(SEQ ID No. 003)
ELKTPLGDTTHTCPRCPEPKSCDTPPPCPRCPE216PKSCDTPPPCPRC
PAPELLG
IgG4:
(SEQ ID NO. 004)
E216SKYGPPCPSCPAPEFLG.

In the context of the present invention it is referred to amino acid positions in the Fc domain, these amino acid positions or residues are indicated according to the EU numbering system as described, for example in Edelman, G. M. et al., Proc. Natl. Acad. USA, 63, 78-85 (1969).

The term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed because they provide structural features or biological activity that are not required for the bispecific antigen binding proteins of the invention. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

Accordingly, in one embodiment the Fc-domain, such as Fc1 and/or Fc2, comprises a hinge domain.

In one embodiment, the Fc-domain is a human IgG Fc domain, preferably derived from human IgG1, IgG2, IgG3 or IgG4, preferably IgG1 or IgG2, more preferably IgG1.

In some embodiments, in particular, when the bispecific antigen binding protein comprises two Fc domains, i.e. in the TCER® format described herein below (such as Fc1 and Fc2), the two Fc domains may be of the same immunoglobulin isotype or isotype subclass or of different immunoglobulin isotype or isotype subclass, preferably of the same. Accordingly, in some embodiments Fc1 and Fc2, are of the IgG1 subclass, or of the IgG2 subclass, or of the IgG3 subclass, or of the IgG4 subclass, preferably of the IgG1 subclass, or of the IgG2 subclass, more preferably of the IgG1 subclass.

In some embodiments, the Fc domain is a variant Fc domain and thus comprises one or more of the amino acid substitutions described herein below.

In some embodiments, the Fc domain comprises or further comprises the “RF” and/or “Knob-into-hole” mutation, preferably the “Knob-into-hole” mutation.

The “RF mutation” typically refers to the amino acid substitutions of the amino acids HY into RF in the CH3 domain of Fc domains, such as the amino acid substitution H435R and Y436F in CH3 domain as described by Jendeberg, L. et al. (1997, J. Immunological Meth., 201: 25-34) and is described as advantageous for purification purposes as it abolishes binding to protein A. In case the bispecific antigen binding protein comprises two FC-domains, the RF mutation may be in one or both, preferably in one Fc-domain.

The “Knob-into-Hole” mutation or also called “Knob-into-Hole” technology refers to amino acid substitutions T366S, L368A and Y407V (Hole) and T366W (Knob) both in the CH3-CH3 interface to promote heteromultimer formation. Those knob-into-hole mutations can be further stabilized by the introduction of additional cysteine amino acid substitutions Y349C and S354C. The “Knob-into-Hole” technology together with the stabilizing cysteine amino acid substitutions has been described in U.S. Pat. Nos. 5,731,168 and 8,216,805.

In some cases artificially introduced cysteine bridges may improve the stability of the bispecific antigen binding proteins, optimally without interfering with the binding characteristics of the bispecific antigen binding proteins. Such cysteine bridges can further improve heterodimerization.

Further amino acid substitutions, such as charged pair substitutions, have been described in the art, for example in EP 2 970 484 to improve the heterodimerization of the resulting proteins.

A sequence with “at least 85% identity to a reference sequence” is a sequence having, on its entire length, 85%, or more, in particular 90%, 91%, 92%, 93%, 94%, 95%, 96%, 97%, 98% or 99% sequence identity with the entire length of a reference sequence.

In the context of the present application, the “percentage of identity” is calculated using a global pairwise alignment (i.e. the two sequences are compared over their entire length). Methods for comparing the identity of two or more sequences are well known in the art. The “needle” program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the World Wide Web site and is further described in the following publication (EMBOSS: The European Molecular Biology Open Software Suite (2000) Rice, P. Longden, I. and Bleasby, A. Trends in Genetics 16, (6) pp. 276-277). The percentage of identity between two polypeptides, in accordance with the invention, is calculated using the EMBOSS: needle (global) program with a “Gap Open” parameter equal to 10.0, a “Gap Extend” parameter equal to 0.5, and a Blosum62 matrix.

Proteins consisting of an amino acid sequence “at least 80%, 85%, 90%, 95%, 96%, 97%, 98% or 99% identical” to a reference sequence may comprise amino acid mutations such as deletions, insertions and/or substitutions compared to the reference sequence. In case of substitutions, the protein consisting of an amino acid sequence at least 80%, 85%, 90%, 95%, 96%, 97%, 98% or 99% identical to a reference sequence may correspond to a homologous sequence derived from another species than the reference sequence.

“Amino acid substitutions” may be conservative or non-conservative. Preferably, substitutions are conservative substitutions, in which one amino acid is substituted for another amino acid with similar structural and/or chemical properties.

In an embodiment, conservative substitutions may include those, which are described by Dayhoff in “The Atlas of Protein Sequence and Structure. Vol. 5”, Natl. Biomedical Research, the contents of which are incorporated by reference in their entirety. For example, in an aspect, amino acids, which belong to one of the following groups, can be exchanged for one another, thus, constituting a conservative exchange: Group 1: alanine (A), proline (P), glycine (G), asparagine (N), serine (S), threonine (T); Group 2: cysteine (C), serine (S), tyrosine (Y), threonine (T); Group 3: valine (V), isoleucine (I), leucine (L), methionine (M), alanine (A), phenylalanine (F); Group 4: lysine (K), arginine (R), histidine (H); Group 5: phenylalanine (F), tyrosine (Y), tryptophan (W), histidine (H); and Group 6: aspartic acid (D), glutamic acid (E). In an aspect, a conservative amino acid substitution may be selected from the following of T→A, G→A, A→I, T→V, A→M, T→I, A→V, T→G, and/or T→S.

In a further embodiment, a conservative amino acid substitution may include the substitution of an amino acid by another amino acid of the same class, for example, (1) nonpolar: Ala, Val, Leu, Ile, Pro, Met, Phe, Trp; (2) uncharged polar: Gly, Ser, Thr, Cys, Tyr, Asn, Gln; (3) acidic: Asp, Glu; and (4) basic: Lys, Arg, His. Other conservative amino acid substitutions may also be made as follows: (1) aromatic: Phe, Tyr, His; (2) proton donor: Asn, Gln, Lys, Arg, His, Trp; and (3) proton acceptor: Glu, Asp, Thr, Ser, Tyr, Asn, Gln (see, for example, U.S. Pat. No. 10,106,805, the contents of which are incorporated by reference in their entirety).

“PRAME” or “Preferentially Expressed Antigen In Melanoma” was first identified as an antigen that is over expressed in melanoma (Ikeda et al Immunity. 1997 February; 6(2): 199-208); it is also known as CT130, MAPE, OIP-4 and has the Uniprot accession number P78395 (as available on Jan. 11, 2019). The protein functions as a repressor of retinoic acid receptor signaling (Epping et al., Cell. 2005 Sep. 23; 122(6):835-47). PRAME belongs to the family of germline-encoded antigens known as cancer testis antigens. Cancer testis antigens are attractive targets for immunotherapeutic intervention since they typically have limited or no expression in normal adult tissues. PRAME is expressed in a number of solid tumors as well as in leukemias and lymphomas (Doolan et al Breast Cancer Res Treat. 2008 May; 109(2):359-65; Epping et al Cancer Res. 2006 Nov. 15; 66(22): 10639-42; Ercolak et al Breast Cancer Res Treat. 2008 May; 109(2):359-65; Matsushita et al Leuk Lymphoma. 2003 March; 44(3):439-44; Mitsuhashi et al Int. J Hematol. 2014; 100(1):88-95; Proto-Sequeire et al Leuk Res. 2006 November; 30(11): 1333-9; Szczepanski et al Oral Oncol. 2013 February; 49(2): 144-51; Van Baren et al Br J Haematol. 1998 September; 102(5): 1376-9).

The term “library” as used herein, refers to a diverse collection or population of nucleic acids encoding different polypeptides. Typically, the different polynucleotides in the library are related through, for example, their origin from polynucleotides encoding a TCR and/or an immunoglobulin subunit of a certain type and class e.g., a library might encode variable domains or CDRs derived from an antibody or TCR.

The term “human immune effector cell” as used herein, refers to a cell of the immune system that actively responds to a stimulus. Non-limiting examples of human immune effector cells are T-cells and B-cells. In a preferred embodiment the human immune effector cell is a T-cell. A cell surface antigen of a human immune effector cell is present on the cell surface of the cells and is accessible to be bound by the ABP of the present invention. Typically the cell surface antigen is specific for a certain type of effector cells (e.g. T-cells). Non-limiting examples of cell surface antigens are various components of the T-cell receptor, e.g. TCR-α chain, TCR-β chain, CD3. In a preferred embodiment the cell surface antigen is selected from the group consisting of CD2, CD3 (such as the CD3γ, CD3δ, and CD3ε chains), CD4, CD5, CD7, CD8, CD10, CD11b, CD11c, CD14, CD16, CD18, CD22, CD25, CD28, CD32a, CD32b, CD33, CD41, CD41 b, CD42a, CD42b, CD44, CD45RA, CD49, CD55, CD56, CD61, CD64, CD68, CD90, CD94, CD95, CD117, CD123, CD125, CD134, CD137, CD152, CD163, CD193, CD203c, CD235a, CD278, CD279, CD287, Nkp46, NKG2D, GITR, FcεRI, TCRα/β, TCRγ/δ, HLA-DR and 4-1 BB, or combinations thereof. Combinations thereof refers to complexes of two or more of said antigens, e.g. a TCRα/β CD3 complex. Preferably, the antigen is CD3, a TCRα/β CD3 complex or CD28, more preferably CD3 or a TCRα/β CD3 complex, most preferably CD3.

### EMBODIMENTS

In the following different embodiments of the invention are defined in more detail. Each embodiment so defined may be combined with any other embodiment unless clearly indicated to the contrary. In particular, any feature indicated as being preferred or advantageous may be combined with any other feature or features indicated as being preferred or advantageous.

The present invention provides a method of providing a multispecific antigen binding protein (ABP), wherein the multispecific ABP is comprising at least one T-cell receptor (TCR)-derived binding site, comprising the steps of:
(I) generating a library of nucleic acids encoding multispecific ABPs, which comprises different variants of the multispecific ABPs;
(II) providing a multitude of mammalian cells each expressing one member of the library of step (I) on their cell surface;
(III) contacting the mammalian cells with a first target of the multispecific ABP and selecting a mammalian cell expressing a multispecific ABP binding to the first target;
(IV) optionally contacting the mammalian cell selected in step (III) with a further target of the multispecific ABP and selecting a mammalian cell expressing a multispecific ABP binding to the further target;
(V) isolating a multispecific ABP from the selected mammalian cell of step (III) or if performed step (IV).

The method of the present invention uses multispecific ABPs (preferably bispecific ABPs) comprising at least one TCR-derived binding site in their final format for the maturation of one or more of the binding sites within the ABP. This makes the otherwise required reformatting of the ABP in the final format unnecessary. The maturation of the ABP in its final format typically regards the binding affinity of the multispecific ABP and its binding specificity for its targets (i.e. first target, further target, other target(s)). In a preferred embodiment the multispecific ABP (preferably bispecific ABP) is selected for its binding affinity for its target (preferably with a high binding affinity, i.e. lower Kd value). In a preferred embodiment, the multispecific ABP (preferably bispecific ABP) is selected for its binding specificity for its target. The maturation of the ABP in its final format also allows to identify mutations that are beneficial for other properties of the ABP, such as stability, preferably interdomain stability, solubility, production yield etc. Stability and in particular interdomain stability, can for example be determined by measuring thermal stability of the ABP such as melting temperature analysis. Such properties cannot be identified if the individual binding sites (i.e. TCR derived binding site(s) and/or antibody derived binding site(s)) are matured separately using methods known in the art. Furthermore, the method of the present invention is performed in mammalian cells, which avoids complications arising from using different cell systems in maturation and production as well as verifying that the maturated binding sites show the same effects in mammalian cells.

In a preferred embodiment the ABP selected in step (III) is specifically binding to the first target.

In a preferred embodiment the ABP selected in step (IV), if performed, is specifically binding to the further target.

In a preferred embodiment the method is further comprising the step of (VI) determining at least one property of the isolated multispecific ABP selected from stability (preferably interdomain stability), solubility, production yield and hydrophobic patches. In a particularly preferred embodiment the property determined in the isolated multispecific ABP, preferably bispecific ABP, is stability. In a particularly preferred embodiment the property determined in the isolated multispecific ABP, preferably bispecific ABP, is solubility. In a particularly preferred embodiment the property determined in the isolated multispecific ABP, preferably bispecific ABP, is production yield. In a particularly preferred embodiment the property determined in the isolated multispecific ABP, preferably bispecific ABP, are the hydrophobic surfaces of the ABP. Typically the hydrophic surfaces are determined by hydrophobic interaction chromatography that separates analytes by the interaction of their hydrophobic surfaces with a hydrophobic matrix. In a particular preferred embodiment hydrophobic surfaces are homogenous in the sense that they appear as a homogenous group in hydrophic interaction chromatography analysis.

In a particular preferred embodiment the property determined in step (VI) is homogenous in the sense that the measured property has a very low variation among the measured ABPs.

### Multispecific Antigen Binding Proteins

Multispecific antigen binding proteins (ABPs) of the present invention are polypeptides or binding proteins that are able to specifically bind to multiple antigens. In a preferred embodiment the multispecific ABP is a bispecific ABP, which is able to specifically bind to two antigenic targets.

At least one binding site of the multispecific ABP of the present invention is derived from a TCR. Another source of binding sites in the multispecific ABP are antibodies. Thus a multispecific ABP comprises at least one TCR-derived binding site and may include further TCR-derived or antibody derived binding sites.

In a preferred embodiment, the multispecific ABP is a bispecific ABP comprising a TCR derived and an antibody derived binding site.

In a preferred embodiment the multispecific ABP is a bispecific ABP and the TCR derived binding site is the first binding site and an antibody derived binding site is the second binding site.

In a preferred embodiment the multispecific ABP (preferably a bispecific ABP) comprises at least one antibody-derived binding site.

In a preferred embodiment the multispecific ABP (preferably a bispecific ABP) is a soluble ABP.

In a preferred embodiment the first binding site, preferably a TCR derived binding site, binds to the first target, preferably an MHC-associated peptide epitope, and the second binding site, preferably an antibody derived binding site, binds the further target, preferably a cell surface antigen of a human immune effector cell.

The multispecific ABPs (preferably a bispecific ABP) may, in addition to the binding sites, comprise further elements. Such elements include components present in TCRs and antibodies other than binding sites. Non-limiting examples include constant domains or parts thereof. Preferred elements include Fc domains (and variants thereof) and/or hinge regions (and variants thereof) of antibodies.

In a preferred embodiment, the multispecific ABP (preferably a bispecific ABP) comprises two Fc domains.

In a preferred embodiment, the multispecific ABP (preferably a bispecific ABP) comprises two polypeptide chains, wherein the binding sites are formed by elements present on different polypeptide chains. Preferably the elements forming the binding sites that are located on the same polypeptide chain are linked by a linker, LINK1 or LINK2. In a preferred embodiment LINK1 and/or LINK2 comprises or consists of the sequence selected from GGGS (SEQ ID NO: 005), GGGGS (SEQ ID NO: 006), TVLRT (SEQ ID NO:007), TVSSAS (SEQ ID NO:008), TVLSSAS (SEQ ID NO:009) and GGGSGGGG (SEQ ID NO:010).

A non-limiting example of a preferred multispecific ABP is a bispecific T cell engaging receptor (TCER*) as disclosed in WO2019/012138A1. Preferred bispecific ABPs, including TCERs, comprise two polypeptide chains. Preferably, the first polypeptide chain comprises a variable domain of an antibody and a variable domain of a TCR, that are connected by a linker. The second polypeptide chain comprises the corresponding variable domain of the antibody and the corresponding variable domain of a TCR that are connected by a linker. The variable domains of the TCR and the antibody on the two polypeptide chains can combine to form a TCR derived binding site and an antibody derived binding site. The two polypeptide chains preferably further comprise elements such as hinge domains and/or Fc domains.

In a preferred embodiment the multispecific ABP is a bispecific polypeptide molecule comprising a first polypeptide chain and a second polypeptide chain, wherein:
a) the first polypeptide chain comprises
   ai) a first variable domain (VD1) of an antibody,
   aii) a first variable domain (VR1) of a T cell receptor (TCR), and
   aiii) a first linker (LINK1) connecting said domains;
b) the second polypeptide chain comprises
   bi) a second variable domain (VR2) of a TCR,
   bii) a second variable domain (VD2) of an antibody, and
   biii) a second linker (LINK2) connecting said domains;
      wherein the first variable domain (VR1) is one of a TCR Va domain and a TCR Vβ domain, and the second variable domain (VR2) is the other one of the TCR Vα domain and the TCR Vβ domain, said first variable domain (VR1) and said second variable domain (VR2) associate to form a first binding site (VR1)(VR2) that specifically binds the first target, preferably an MHC-associated peptide epitope;
      wherein said first variable domain (VD1) and said second variable domain (VD2) associate to form a second binding site (VD1)(VD2) that specifically binds the further target, preferably a cell surface antigen of a human immune effector cell;
      wherein said two polypeptide chains are fused to human IgG hinge domains and/or human IgG Fc domains or dimerizing portions thereof;
      wherein said two polypeptide chains are connected by covalent and/or non-covalent bonds between said hinge domains and/or Fc-domains;
      wherein said dual specificity polypeptide molecule is capable of simultaneously binding the cell surface molecule and the MHC-associated peptide epitope; and
      wherein the order of the variable domains in the two polypeptide chains is selected from VD1-VR1 and VR2-VD2 or VD2-VR2 and VR1-VD1.

In a preferred embodiment the linker-sequences LINK1 and/or LINK2 having a length of 6 to 12 amino acids, preferably 7 to 11 or 8 to 10 amino acids, more preferably 7 to 9 amino acids, most preferably 8 amino acids.

In a preferred embodiment the multispecific ABP is a bispecific ABP (preferably a TCER) wherein the linker-sequences LINK1 and/or LINK2 having a length of 6 to 12 amino acids, preferably 7 to 11 or 8 to 10 amino acids, more preferably 7 to 9 amino acids, most preferably 8 amino acids.

### Targets of the Multispecific ABP

The multispecific ABP of the present invention is intended to bind specifically to multiple targets with its multiple binding sites, i.e. each binding site binds specifically to one target.

In a preferred embodiment the target(s) (i.e. first target, further target(s) and/or other target(s)) of the multispecific ABP is/are peptides. Preferred examples of peptide targets are MHC-associated peptide epitopes (preferably MHC class I associated peptide epitopes), surface antigens of cells (preferably human immune effector cells).

In a preferred embodiment the first target of the multispecific ABP (preferably a bispecific ABP) is a peptide.

In a preferred embodiment the further target of the multispecific ABP (preferably a bispecific ABP) is a peptide.

In a preferred embodiment the other target of the multispecific ABP is a peptide.

In a preferred embodiment the first target of the multispecific ABP (preferably a bispecific ABP) is a MHC-associated peptide epitope, preferably a MHC class I associated peptide epitope.

In a preferred embodiment the further target of the multispecific ABP (preferably a bispecific ABP) is a MHC-associated peptide epitope, preferably a MHC class I associated peptide epitope.

In a preferred embodiment the further target of the multispecific ABP (preferably a bispecific ABP) is a cell surface antigen, preferably of a human immune effector cell.

In a preferred embodiment the first target of the multispecific ABP (preferably a bispecific ABP) is a cell surface antigen, preferably of a human immune effector cell.

ABP-based therapeutics require the ability to provoke a strong, but at the same time highly specific response against the intended target. The assessment of cross-reactivity with variants of the intended target is of high importance to minimize potential safety risks in the therapeutic use. In a preferred embodiment the method also comprises a step (IIIA) which comprises contacting the selected mammalian cell of step (III) with a variant of the first target and selecting a mammalian cell expressing a multispecific ABP (preferably a bispecific ABP) binding to the variant of the first target with a lower binding affinity than binding to the first target. Said variant of the first target is typically an antigenic target (preferably a peptide, more preferably a MHC-associated peptide epitope) that is structurally similar to the first target. For example, a variant of the first target differs by 1, 2, 3, 4, 5, 6, 7, 8 or 9, preferably 1, 2 or 3, more preferably 2 or 3, most preferably 3 amino acids from the first target. In some embodiments the variants of the first target are not identical in length to the first target (i.e. differ in length e.g. by 1 or 2 amino acids).

Variants of the first target may be found in an organism in a different context (e.g. different type of cell, healthy vs disease affected tissue) than the first target and is therefore not the intended target of the multispecific ABP, i.e. an ‘off-target’. In a preferred embodiment the variant of the first target has an amino acid sequence that is at least 60%, 65%, 70%, 75%, 80%, 85%, 90% or 95%, preferably at least 90% or 80%, more preferably at least 90%, identical to the first target. In a preferred embodiment the variant of the first target has an amino acid sequence that is at least 60%, preferably at least 65% identical to the first target. In a preferred embodiment the variant of the first target has an amino acid sequence that is less than 60%, 50%, 40% preferably less than 40%, identical to the amino acid sequence of the first target. Variants of the first target expressed in a mammal, preferably a human, are of particular relevance. Variants of the first target are of particular relevance if they are indeed expressed in the organism to be eventually treated with the multispecific ABP of the present invention. Binding to the variant would then result in an ‘off-target effect’. Typically an ‘off-target effect’ is of relevance if the variant of the first target is expressed in healthy, preferably non-cancerous, tissue.

In a preferred embodiment step (IIIa) is performed simultaneous with step (III). This allows for the simultaneous selection for target binding and counterselection for off target binding, thereby preserving the binding specificity of the ABP during the affinity maturation process. In a preferred embodiment the first target in step (III) is labeled with a first label and the variant of the first target in step (IIIa) is labelled with a second label. Any labels that can be discriminated by using standard methods such as flow cytometry can be used. In a preferred embodiment, in step (IIIa) more than one variant of the first target is used and the additional variants are labelled with different labels.

In a preferred embodiment the method also comprises a (preferably optional) step (IVa) which comprises contacting the selected mammalian cell of step (IV) with a variant of the further target and selecting a mammalian cell expressing a multispecific ABP (preferably a bispecific ABP) binding to the variant of the further target with a lower binding affinity than binding to the further target. Said variant of the further target is typically an antigenic target (preferably a peptide, more preferably a cell surface antigen, most preferably a cell surface antigen of a human immune effector cell) that is structurally similarly to the further target. For example a variant of the further target differs by 1, 2, 3, 4, 5, 6, 7, 8 or 9, preferably 1, 2 or 3, more preferably 2 or 3, most preferably 3 amino acids from the further target. Such variants of the further target may be found in an organism in a different context (e.g. different type of cell, healthy vs disease affected tissue) than the further target and is therefore not the intended target of the multispecific ABP, i.e. an ‘off-target’. In a preferred embodiment the variant of the further target has an amino acid sequence that is at least 30%, 40%, 50%, 60%, 65%, 70%, 75%, 80%, 85%, 90% or 95%, preferably 90%, identical to the further target. In a preferred embodiment the variant of the further target has an amino acid sequence that is at least 60%, preferably at least 65% identical to the further target. In a preferred embodiment the variant of the further target has an amino acid sequence that is less than 60%, 50%, 40% preferably less than 40%, identical to the amino acid sequence of the further target. Variants of the further target expressed in a mammal, preferably a human, are of particular relevance. Variants of the further target are of particular relevance if they are indeed expressed in the organism to be eventually treated with the multispecific ABP of the present invention in cell types or tissues not intended to be targeted. Binding to the variant would then result in an ‘off-target effect’. For example, in a preferred embodiment the further target is a cell surface antigen, preferably of a human immune effector cell, and the variant of the further target would be expressed in another cell type or tissue of the organism.

In a preferred embodiment step (IVa) is performed simultaneous with step (IV).

In an embodiment the method also comprises a (preferably optional) step (IVb), which comprises repeating steps (IV) and optionally step (IVa) for 1, 2, 3 or 4 other targets and respectively variants of the multispecific ABP. This embodiment is in particular relevant for a multispecific ABP with more than two binding specificities, i.e. 3, 4, 5 or more binding specificities. Said variant of the other target is typically an antigenic target (preferably a peptide, more preferably a cell surface antigen, most preferably a cell surface antigen of a human immune effector cell) that is structurally similarly to the other target. For example a variant of the other target differs by 1, 2, 3, 4, 5, 6, 7, 8 or 9, preferably 1, 2 or 3, more preferably 2 or 3, most preferably 3 amino acids from the other target. Such variants of the other target may be found in an organism in a different context (e.g. different type of cell, healthy vs disease affected tissue) than the further target and is therefore not the intended target of the multispecific ABP, i.e. an ‘off-target’. In a preferred embodiment the variant of the other target has an amino acid sequence that is at least 30%, 40%, 50%, 60%, 65%, 70%, 75%, 80%, 85%, 90% or 95%, preferably 90%, identical to the other target. In a preferred embodiment the variant of the other target has an amino acid sequence that is at least 60%, preferably at least 65% identical to the other target. In a preferred embodiment the variant of the other target has an amino acid sequence that is less than 60%, 50%, 40% preferably less than 40%, identical to the amino acid sequence of the other target. Variants of the other target expressed in a mammal, preferably a human, are of particular relevance. Variants of the other target are of particular relevance if they are indeed expressed in the organism to be eventually treated with the multispecific ABP of the present invention. Binding to the variant would then result in an ‘off-target effect’.

In a preferred embodiment the multispecific ABP (preferably a bispecific ABP) binds (preferably with a TCR derived binding site) to the first target (preferably an MHC-associated peptide epitope) with a binding affinity of between 1 pM and 100 nM, 10 pM to 90 nM, 20 pM to 80 nM, 30 pM to 70 nM, 40 pM to 60 nM, 50 pM to 50 nM, 60 pM to 40 nM, 70 pM to 30 nM, 80 pM to 20 nM, 90 pM to 10 nM, 1 nM to 10 nM, 1 nM to 5 nM preferably 90 pM to 10 nM, 1 nM to 10 nM, 1 nM to 5 nM, more preferably 1 nM to 10 nM, 1 nM to 5 nM, most preferably 1 nM to 10 nM.

In a preferred embodiment the multispecific ABP (preferably a bispecific ABP) binds (preferably with an antibody derived binding site) to the further target (preferably a cell surface antigen, more preferably a cell surface antigen of a human immune effector cell) with a binding affinity of between 1 nM to 5 μM, 5 nM to 3 μM, 10 nM to 2 μM, preferably 15 nM to 1 μM.

In a preferred embodiment the multispecific ABP (preferably a bispecific ABP) binds (preferably with an antibody derived binding site) to the further target (preferably a cell surface antigen, more preferably a cell surface antigen of a human immune effector cell) with a monovalent binding affinity of between 1 nM to 5 μM, 5 nM to 3 μM, 10 nM to 2 μM, preferably 15 nM to 1 μM.

In a preferred embodiment the multispecific ABP (preferably a bispecific ABP) binds (preferably with a receptor or ligand derived binding site) to the further target (preferably a cell surface antigen, more preferably a cell surface antigen of a human immune effector cell) with a binding affinity of between 500 pM and 1000 nM, 1 nM and 800 nM, 10 nM and 500 nM, 50 nM and 400 nM, 100 nM and 300 nM, preferably 100 nM to 200 nM. In a preferred embodiment the receptor or ligand derived binding site has more than one binding site and thus has a binding avidity with an affinity of between

In a preferred embodiment the multispecific ABP (preferably a bispecific ABP) binds to the first target (preferably a MHC-associated peptide epitope) with higher affinity (i.e. a lower Kd value) than to the further target (preferably a cell surface antigen, more preferably a cell surface antigen of a human immune effector cell).

In a preferred embodiment the multispecific ABP (preferably a bispecific ABP) binds to the variant of the first target (i.e. an ‘off-target’) with a binding affinity of less than 100 nM.

In a preferred embodiment the multispecific ABP (preferably a bispecific ABP) binds to the variant of the first target (i.e. an ‘off-target’) with a binding affinity two orders of magnitude lower than the first target.

In a preferred embodiment the multispecific ABP (preferably a bispecific ABP) binds to the variant of the first target (i.e. an ‘off-target’) with the same or higher binding affinity as to the first target. In such embodiments the variant of the first target

In a preferred embodiment the multispecific ABP (preferably a bispecific ABP) binds to the variant of the further target (i.e. an ‘off-target’) with a binding affinity of less than 10 μM.

In a preferred embodiment the multispecific ABP (preferably a bispecific ABP) binds to the variant of the further target (i.e. an ‘off-target’) with a binding affinity two orders of magnitude lower than the further target.

In a preferred embodiment the multispecific ABP (preferably a bispecific ABP) binds to the variant of the other target (i.e. an ‘off-target’) with a binding affinity of less than 10 μM.

In a preferred embodiment the multispecific ABP (preferably a bispecific ABP) binds to the variant of the other target (i.e. an ‘off-target’) with a binding affinity two orders of magnitude lower than the other target.

In a preferred embodiment the multispecific ABP (preferably a bispecific ABP, more preferably a TCER format) obtained by the method of the invention binds simultaneously to an MHC-associated peptide epitope on a cancerous cell and a cell surface antigen of a human immune effector cell and thereby mediates cytotoxic effects on the cancerous cell with an EC₅₀ value of 100 fM to 100 nM, 1 pM to 10 nM, 5 pM to 8 nM, 20 pM to 5 nM, 30 pM to 3 nM, 40 pM to 2 nM, 50 pM to 1 nM, preferably 50 pM to 1 nM.

In a preferred embodiment the cytotoxic effect on cancerous cells is determined by measuring LDH-release. An example of an LDH-release assay is disclosed in the experimental section of the present specification.

In a preferred embodiment the multispecific ABP (preferably a bispecific ABP, more preferably a TCER format) obtained by the method of the invention binds simultaneously to an MHC-associated peptide epitope on a cancerous cell and a cell surface antigen of a human immune effector cell and thereby mediates cytotoxic effects on the cancerous cell with an EC₅₀ vale of at least 100 fM, 1 pM, 10 pM, 50 pM, 60 pM, 80 pM, 100 pM, 150 pM, 200 pM, 250 pM preferably at least 50 pM.

### Libraries

The method of the present invention uses display libraries that allow the expression of multispecific ABPs (preferably bispecific ABPs) on the surface of mammalian cells. These libraries contain a multitude of nucleic acids that encode different variants of the multispecific ABPs. Said variant of the ABP differs in the amino acid sequence from the ABP. In a preferred embodiment the sequence identity between the ABP and the variant of the ABP is at least 60%, 70%, 80%, 85%, 90%, 95%, 96%, 97%, 98%, 99%, >99%, preferably 90%, 95%, 99%.

In a preferred embodiment the variant of the ABP differs by the exchange of complete CDRs (i.e. CDR1, CDR2 or CDR3).

In a preferred embodiment these differences are in the binding site of the ABP. The differences can occur in the CDR regions of TCR-derived and antibody derived binding sites and/or in other part s of the variable domains. In a preferred embodiment the sequence identity between the ABP and the variant of the ABP in the binding site (preferably the TCR derived binding site) is 80%, 85%, 90%, 95%, 96%, 97%, 98%, 99%, >99%, preferably 90%, 95%, 99%.

In a preferred embodiment the differences are outside the binding site of the ABP. These differences typically affect other properties than binding affinity, such as stability, solubility, production yield.

In a preferred embodiment the differences are in the binding site of the ABP as described above and in other parts of the ABP as described above. Such differences typically occur if the binding affinity and other properties of the ABP are improved. Improved binding affinity and specificity is typically the result of sequence changes in the CDR or framework regions of the ABP.

In another preferred embodiment the differences may occur anywhere in the ABP and regard the removal or deactivation of disadvantageous sequence elements selected from N-glycosilation sites, deamidation sites, isomerization sites, oxidation sites and protease cleavage sites.

The method of the invention can be used with libraries of different sizes. Examples of library sizes range from up to 10⁴ to up to 10¹¹ members, i.e. up to 10⁴, 10⁵, 10⁶, 10⁷, 10⁸, 10⁹, 10¹⁰, 10¹¹. In a preferred embodiment the library has a size of up to 10⁸.

There are a variety of techniques known in the art that may be used to efficiently generate libraries usable in the present invention including those described or referenced in Molecular Cloning—A Laboratory Manual, 3.sup.rd Ed. (Maniatis, Cold Spring Harbor Laboratory Press, New York, 2001), Current Protocols in Molecular Biology (John Wiley & Sons), each of which is incorporated by reference in its entirety. Such methods include but are not limited to gene assembly methods, PCR-based method and methods, which use variations of PCR, ligase chain reaction-based methods, pooled oligo methods such as those used in synthetic shuffling, error-prone amplification methods and methods which use oligos with random mutations, classical site-directed mutagenesis methods, cassette mutagenesis, and other amplification and gene synthesis methods. A variety of commercially available kits and methods for gene assembly, mutagenesis, vector subcloning, and the like, are available for generating nucleic acids that encode immunoglobulin amino acid sequences.

In a preferred embodiment the library is created by a CDR combinatorial approach.

In a preferred embodiment the library is created by providing a pool of known CDR sequences of a TCR or antibody that are freely combined in every conceivable way. Such libraries may be further diversified by randomly mutating one or more amino acid in the CDRs thereby creating variants of the CDRs. Preferred methods for random mutagenesis are error prone PCR, the use of degenerated primers, chain shuffling libraries, libraries from fusion PCRs, synthetic libraries and golden gate cloning. In a more preferred embodiment the library is a completely synthesized library.

In a preferred embodiment the library encoding multispecific ABPs (preferably bispecific ABPs) is created by somatic hypermutation.

In a preferred embodiment the library results from a previous maturation step of a multispecific ABP or of multiple ABPs with single specificity. Preferably the previous maturation step was performed in yeast cells.

In a preferred embodiment the multispecific ABPs (preferably bispecific ABPs) encoded in the library are fused to a transmembrane domain. The transmembrane domain allows the expression of a soluble multispecific ABP (preferably bispecific ABP) on the cell surface of a mammalian cell (preferably a CHO cell). Preferred examples of a transmembrane domain are a PDGFR transmembrane domain or murine H-2Kk domain, preferably a PDGFR transmembrane domain.

### Mammalian Cells

The present invention discloses a mammalian cell display platform for targeted expression and engineering of therapeutic proteins, in particular multispecific ABPs (preferably bispecific ABPs). The method of the present invention can in principle use any mammalian cell able to express the multispecific ABP. Typically the multispecific ABP (preferably bispecific ABP) is expressed on the surface of the mammalian cell to allow the ABP to contact with a first target and optionally with further target(s).

In a preferred embodiment the mammalian cell is selected from Chinese hamster ovary cells (CHO) cells or human embryonic kidney cells (HEK) cells, preferably CHO cells.

Further examples of suitable mammalian host cells include HeLa cells (HeLa S3 cells, ATCC CCL2.2), Jurkat cells, Raji cells, Daudi cells, human embryonic kidney cells (293-HEK; ATCC 293c18, ATCC CRL 1573), African green monkey kidney cells (CV-1; Vero; ATCC CRL 1587), SV40-transformed monkey kidney cells (COS-1; ATCC CRL 1650), canine kidney cells (MDCK; ATCC CCL 34), baby hamster kidney cells (BHK-21, BHK-570; ATCC CRL 8544, ATCC CRL 10314), Chinese hamster ovary cells (CHO-K1; ATCC CCL61; CHO DG44 (Chasin et al., 1986, Som Cell Molec Genet, 12, 555)), and other rodent cell lines such as NSO, SP2/O, GH1 (ATCC CCL82), H-4-II-E (ATCC CRL 1548), NIH-3T3 (ATCC CRL 1658).

In a preferred embodiment the mammalian cell used in the method of the present invention is identical to the mammalian cell used for mid-scale or large-scale production of the multispecific ABP. Typically mammalian cells (preferably CHO cells) tend to express mutations identified in these cells (preferably CHO cells) better than mutations found in other mammalian host cells (Chen et al., Biotechnol Bioeng 2016; 113:39-51). Thus the use of the same mammalian cells in the method of the present invention and the later production stage of the ABPs is beneficial.

In a preferred embodiment the mammalian cells (preferably CHO cells) are genetically modified to allow the repeated modification of the cells. Preferably the modification allows for site-specific recombination processes. In a preferred embodiment mammalian cells (preferably CHO cells) are employed that are genetically modified to contain a landing pad allowing for RMCE-mediated integration of genetic elements encoding for biomolecules. Genetic information integrated into the mammalian cells (preferably CHO cells) by site-specific recombination processes are expressed from a single copy at the same genetic locus. Such a targeted single copy integration has significant advantages over display systems based on standard transfection, viral transduction, and transposases, which often result in incorporation of multiple copies at different integration sites leading to variations in the protein expression levels making subsequent screening and selection processes more complicated.

In a preferred embodiment the modified mammalian cells (preferably CHO cells) contain a pre-defined locus flanked with Flp recombination target (FRT) sites for Flp recombinase recognition.

In a preferred embodiment the modified mammalian cells (preferably CHO cells) is stable cell line.

In a preferred embodiment the use of modified mammalian cells (preferably CHO cells) using RMCE-mediated integration of genetic elements results in long-term expression stability of the integrated elements. Stable expression is achieved for at least 120 days, 135 days, 150 days, 175 days or 200 days, preferably for at least 135 days.

In a preferred embodiment an electroporation protocol is applied to modify the mammalian cells (preferably CHO cells).

In a preferred embodiment an electroporation protocol is applied in which RNA encoded Flp recombinase together with the DNA encoded donor vector is used for mammalian cell (preferably CHO cell) transfection. This results in exchange rates of about 5%, which is comparable to exchange efficiencies achieved with alternative (but technically more complicated) technologies designed for targeted protein engineering like CRISPR/Cas9 and TALEN showing also up to 5% exchange efficiency (Parthiban et al, Mabs 2019; 11:884-98; Parola et al, Mabs 2019; 11:1367-80). In a preferred embodiment an enhanced Flp version (see Phan et al, Sci Rep-uk 2017; 7:17771) is used. Thus the exchange efficiency rates can be increased to more than 7%.

In a preferred embodiment this is combined with a MaxCyte flow electroporation system that enables electroporation of up to 1×10¹¹ cells in a 30-min cycle. In particular in combination with high exchange rates (i.e. 5% or 7%) this allows generation of large mammalian display libraries with a size of about 1×10⁹.

### EXAMPLES

An improved maturation process of TCR-based therapeutics is disclosed herein utilizing a maturation platform based on mammalian cells allowing TCR- and antibody derived binding site maturation in the context of a final multispecific TCR format. The disclosed system is based on a stable CHO cell line with a high expression landing pad in which biomolecules can be integrated by recombinase-mediated cassette exchange (RMCE). RMCE enables the efficient and directed insertion of a DNA fragment into a single copy, predefined locus flanked with Flp recombination target (FRT) sites for Flp recombinase recognition.

Using a model TCR targeting an HLA-A*02-presented peptide derived from preferentially expressed antigen in melanoma (PRAME), the inventors demonstrate that the CHO based system can be employed for the maturation of a bispecific T cell engaging receptor (TCER) format, comprising a TCR and an antibody derived binding site. Therefore, a CHO library of TCER molecules was generated comprising thousands of TCR variants encoding different combinations of previously identified complementarity determining region (CDR) mutations in all six CDRs of the model PRAME TCR. The disclosed system allows for simultaneous selection for target binding and counterselection for off target binding, thereby preserving the binding specificity of the TCR during the affinity maturation process. From the selected TCER candidates the inventors characterized 10 candidates in more detail to demonstrate their ability to recognize PRAME peptide HLA (pHLA) at a physiologically relevant level. The candidates showed strong induction of anti-tumor cell responses as measured by lysis of tumor cell lines and a robust activation of T cells supporting the use of the CHO display system for targeted engineering of bispecific TCR-based molecules.

### Material and Methods Used

### Cell Culture

CHO-S cells (Thermo Fisher Scientific, Waltham, USA) were cultured in ActiPro (Cytiva, #SH31039.02) with 1×HT (Gibco, #11067030) and 8 nM GlutaMax (Gibco, #35050061). 1 mg/mL Geneticin (Gibco, #10131027) was added to apply selection pressure. CHO cells were cultured in suspension in a 50 mm orbital shaker at 95 rpm (flasks) or 145 rpm (50 mL cell reactors) with 5% CO₂, 37° C., and 70% relative humidity (rH). Cells were subcultured every 2-3 days.

After single cell sorts CHO cells were cultured in 100 μL CD CHO (Gibco, #10743029) with 1:20 InstiGRO CHOPLUS (Solentim Ltd, #RS-1225) in a 96-well flat bottom plate and incubated for 10-14 days with a gas permeable adhesive film at 37° C., 5% CO₂, and 80% rH without shaking.

All tumor cell lines were cultured at 5% CO₂, 37° C., and 80% rH and were passaged at a confluency above 80% (adherent) or every 2-3 days (suspension). Adherent cells were detached using 0.05% Trypsin/EDTA (Gibco, #11580626) or Accutase (PromoCell, #C-41310), centrifuged, and resuspended in new culture medium. Suspension cells were centrifuged and transferred into new medium. UACC257 cells⁴⁰ were cultured in RPM11640 (Gibco, #A10491-01) supplemented with 15% FCS (Life Technologies, #10270106). DMEM high glucose medium (Gibco, #31966-021) with 10% FCS and 1×MEM NEAA (Gibco, #11140050) was used for HS695T cells.⁴¹ A375 cells⁴² required DMEM high glucose medium with 10% FCS. SET-2 cells⁴³ were cultured in RPM11640 and 20% FCS.

### PBMC Isolation and Freezing

Peripheral blood mononuclear cells (PBMCs) from HLA-A*02 positive healthy donors were isolated from leukapheresis products obtained from the DRK Mannheim. In brief, blood samples were directly diluted 1:1 with DPBS (Gibco, #14190250) and carefully layered over 15 mL of Pancoll (PAN Biotech, #P04-60500) and centrifuged for 20 min at room temperature (RT) at 800×g without brake. The obtained PBMC layer was washed with DPBS by centrifugation (300×g, RT, 10 min). The cell pellet was resuspended in DPBS with 1% human serum albumin (HSA) (OctaPharma, #PZN-00200331) and cells were counted using the ViCell XR device (Beckmann Coulter, Brea, USA). For freezing, the supernatant was discarded and cells were resuspended in RPM11640, 11.5% HSA. RPM11640, 11.5% HSA, 20% DMSO (WAK Chemie, #WAK-DMSO-70) was added dropwise (v/v 1:1) directly before freezing cells in a CellCool FTS30 container and stored at −80° C. prior to transferring the cells into liquid nitrogen. PBMCs were thawed in a water bath at 37° C. Cell suspension was transferred into CTL-Wash buffer (C.T.L., #CTLW-10) supplemented with 0.1% L-Glutamine (Gibco, #25030-024) and 50 U/mL Benzonase (VWR, #1.01695.0001) before centrifugation and resuspension in RPMI GlutaMax (Gibco, #72400021) with 10% human serum (heat inactivated) (c.c. pro, Oberdorla, Germany), 1% Penicillin/Streptomycin (Biozym, #DE17-602), 20 μg/mL Gentamycin (Lonza, #17-15197), and 1% sodium pyruvate (c.c. pro, #Z-20-M) supplemented with 10 U/mL Interleukin-2 (IL-2).

### Functional Assays

Adherent target cells were plated one day before the co-culture start in a 96-well flat bottom plate. For even distribution plates were shaked for 30 s at 450 rpm on an orbital shaker and incubated afterwards at 37° C., 5% CO₂, and 80% rH in the respective cell line medium. Effector cells were thawed at the same day. The next day, medium from the adherent target cell line was removed and 50 μL/well assay medium (RPMI 1640 without phenol red (Gibco, #11835-063), 10% heat-inactivated human serum, 1×GlutaMax (Gibco, #35050-038), 1×Penicillin/Streptomycin) supplemented with 10 U/mL IL-2 was added. Suspension cells were seeded at the day of the co-culture start. Cells were harvested by centrifugation at 300×g for 5 min and resuspended in the assay medium. Suspension cells were seeded into 96-well round bottom plates. Serial dilutions of TCER molecules were performed in the assay medium for final concentrations of 10 nM-0.01 pM. Effector cells were harvested and resuspended in the assay medium and added to each well. An effector to target ratio (E:T) of 10:1 was applied for all co-cultures. After 48 h incubation, the supernatant was either used for the assessment of target cell killing or for the measurement of cytokine levels. The CytoTox 96 non-radioactive cytotoxicity assay kit (Promega, #G1780) was used to measure the ability of the TCER molecules to mediate lysis of tumor cells. The experiments were performed following the manufacturer's instructions. The readout was executed using a SpectraMax ix3 (Molecular devices, San José, USA). Cytokine release assays were performed using the MACSPlex cytotoxicity T/NK cell kit (Miltenyi Biotec, #130-125-800) following the manufacturer's instruction. Analysis was performed with the MACSQuant X using the express mode in the MACSQuantify Software (version 2.13.2) (Miltenyi Biotec, Bergisch Gladbach, Germany). A CD3 antibody (BioLegend, #317301) and a CD28 antibody (University of Tuebingen) were used for the positive controls.

### Stable CHO Cell Line Generation

CHO cells were seeded one day before the electroporation at a density of 1.5×10⁶ cells/mL. For electroporation the cells were harvested and washed once with MaxCyte electroporation buffer. After the washing step, cells were resuspended in MaxCyte buffer at a density of 2×10⁸ cells/mL and pJD1_GFP vector DNA was added at final concentration of 1 μg/1×10⁶ cells following linearization via Pvul. The transfection was performed using a MaxCyte STx system (MaxCyte, Gaithersburg, USA) using the CHO-2 program. After resting for 40 min at 37° C., 5% CO₂ without shaking, the cells were resuspended in culture medium at a density of 4×10⁶ cells/mL. The next day, cells were split to a density of 0.25×10⁶ cells/mL in culture medium with 1 mg/mL Geneticin. Cells were selected for 2 weeks for stable GFP expression and the top 2% GFP expressing cells were sorted twice after the expansion of stable clones using an MA800 device (Sony, San José, CA, USA). High GFP expressing cells underwent an RMCE step exchanging GFP for RFP. Therefor the cells were treated as described above and the pJD1_RFP donor vector (1 pg/1×10⁶ cells) and the Flp recombinase supplied as RNA (Miltenyi Biotec, #130-106-769) (4 pg/1×10⁶ cells) were added prior to electroporation. Transfected cells were sorted for GFP negative/RFP positive cells and single cell sorting was performed to screen for stable single copy integration. Single cell clones were sent for targeted locus amplification (Cergentis, Utrecht, Netherlands) and an appropriate clone was expanded as the new landing pad containing CHO cell line.

### TCER Library Generation and Affinity Maturation

CDR variants were obtained from a previous yeast display maturation campaign. Separate CDRs were combined in a single library, which was ordered from GeneArt (Regensburg, Germany). Expression of each chain was controlled by an hCMV promotor. TCER library was generated by transfection of the library DNA in combination with the Flp-encoding RNA. The TCER expressing cells were sorted and expanded. Therefore, the first selection round was performed with 100 nM biotinylated PRAME pHLA (pHLA complexes were prepared as described in the section “Generation of pHLA complexes” below) followed by staining with Streptavidin-APC (Invitrogen, #S868). The second selection round was performed with 31.6 nM PRAME pHLA and 10 nM each of 11 UV-exchanged similar peptide-HLA tetramers, coupled to PE (TET-PE). Finally, a single cell sort was performed using 10 nM PRAME pHLA and 10 nM of each similar pHLA TET-PE. For sorting an MA800 in targeted mode and for analyses an SA3800 (Sony, San José, CA, USA) were used.

For staining cells were washed once in DPBS, 1% BSA, 2 nM EDTA (Carl Roth, #8040.3) and incubated in the first staining solution for 30 min in the dark at 4° C., washed twice and were again incubated for 30 min in the dark at 4° C. with the second staining solution. Before measurements cells were washed once again and resuspended in DPBS, 1% BSA, 2 nM EDTA.

Genomic information of single clones was extracted using the Phire Tissue Direct PCR Master Mix (Thermo Fisher, #F170L) following the instructions for the Dilution & Storage protocol of the manufacturer. The amplification was performed using the primers JD061 (5′-GACACGAAGCTGGCTAG-3′; SEQ ID No. 011) and JD065 (5′-CTTGCTGGCAGAAGTAGG-3′; SEQ ID No. 012) for the alpha chain CDRs of the TCR and JD057 (5′-CAGCGCCTACTCTGAGG-3′; SEQ ID No. 013) and JD059 (5′-GCTGGATATCTGCAGAATTCC-3′; SEQ ID No. 014) for the beta chain. Amplicons were sent to Microsynth Seqlab GmbH (Goettingen, Germany) for sequencing.

### Plasmid Preparation

### Vectors

Different plasmids were created for the generation of a stable CHO cell line expressing a gene of interest from a stably integrated landing pad. The GFP and RFP containing vectors for the landing pad integration were obtained via digestion of pJD1 (in-house construct with standard genetic elements) with NheI (New England Biolabs, #R313M) as well as EcoRI (New England Biolabs, #R3101M) and subsequent cloning resulting in pJD1_GFP and pJD1_RFP, respectively. The pJD1 vector contains FRT and FRT F3 site to allow for an Flp-based exchange of the landing pad. The vector and the GFP and RFP inserts were synthesized by Genscript (Piscataway Township, NJ, USA).

### Transformation and Plasmid Isolation

For the transformation One Shot TOP10 electrocompetent Escherichia coli (E. coli) cells (Thermo Fisher, #C404052) were used following the manufacturer's instructions. After incubation at 37° C. for 24 h single clones were picked and expanded in LB broth (Carl Roth, #X964.1) with 100 μg/mL Ampicillin (Carl Roth, #K029.1). The NucleoBond Xtra Midi (Macherey-Nagel, #740422.50) or Maxi EF Kit (Macherey-Nagel, #740424.50) were used for plasmid DNA isolation following the manufacturer's instructions. Isolated DNA was heated for 20 min at 95° C. and handled sterile afterwards. A NanoDrop 8000 (Thermo Fisher, Waltham, USA) was used to determine the DNA concentrations via absorbance measurement at 260 nm.

### Soluble TCER Expression

Transient transfection was performed using the CHO program of the MaxCyte STx system. The TCER chains were supplied as separate DNA constructs cloned into expression vectors pMH1 (in-house construct) at a 1:1 ratio with 1.5 μg/1×10⁶ cells. Cells were washed once with the MaxCyte electroporation buffer. Cells and DNA were mixed and transferred into an OC-400 cuvette. Afterwards, cells transfected with the same construct were pooled and rested in a 25 cm² cell culture flask at 37° C., 70% rH, and 5% CO₂. After resting, cells were transferred into a shake flask at a density of 4×10⁶ cells/mL. After 24 h of incubation, temperature was reduced from 37° C. to 32° C. and sodium butyrate (Sigma Aldrich, #B5887) was added to a final concentration of 1 mM. Feeding of the cultures occurred at day 4, 6, and 8 with Cell Boost 7A (Cytiva, #SH31119.01) and 7B (Cytiva, #SH31120.01), 5% and 0.5%, respectively. Cells were harvested either at day 11 or when the viability dropped under 70%. Cell supernatants were filtered using SartoClear Dynamics Lab V kits (Sartorius, #SDLV-0500-20E0-E). A final concentration of 0.1% sodium azide (Merck, #1.06688.0025) was added and supernatants were stored at 4° C. until purification. Soluble expressed TCER molecules were purified using tandem purification with a ProteinL and size-exclusion column installed in an Aekta Pure 25 system (Cytiva, Malborough, USA). After elution of Protein L-bound protein at pH 2.8, the protein was directly applied onto a Superdex 200 μg column equilibrated in DPBS. The eluting fractions containing monomeric TCER molecules were collected and pooled. For soluble expression of the parental TCR, a single chain TCR construct containing 4 stabilizing TCR framework mutations was generated and expressed in fusion with the N-terminus (heavy chain) of a Fab fragment of the UCHT1 anti-CD3 antibody. Protein concentration was determined using a NanoDrop 8000 and adjusted to 1 mg/ml either by using Amicon Ultra-15 centrifugal concentrators or by dilution with DPBS. Final protein concentration was determined after 0.22 μm filtration.

Generation of pHLA Complexes

HLA molecules were produced in E. coli as inclusion bodies, purified and refolded essentially as described by Garboczi et al. (Proc National Acad Sci 1992; 89:3429-33). The β₂m molecules and the HLA chains were transferred from the HLA injection into the refolding buffer via a syringe. In the next step, PRAME or a UV-light sensitive peptide was added with a final concentration of 30 μM. This reaction stirred for 2-4 days at 10° C. using ultrafiltration stirred cells with a 30 kDa membrane. Further purification was performed via SEC chromatography with TSBA buffer (DPBS, 2% FCS, 2 mM EDTA, 0.01% sodium azide) and a HiLoad 26/600 75 pg column in an AKTA Prime plus. Protease inhibitor PSMF (Sigma Aldrich, #P7625), leupeptin (Roche, #11017101001), and pepstatin (Roche, #11359053001) were added. The mixture was concentrated to 2000 μg/mL via an Amicon Ultra-15 centrifugation unit. The biotinylation process was conducted overnight via BirA biotin-protein ligase (Avidity LCC, #Bulk-BirA) at 27° C. following the manufacturer's instruction. The pHLA complexes were again gel-filtered, concentrated, and aliquoted.

All target and off-target peptides (Table 1) used were produced in-house via a standard Fmoc chemistry with a Syro II synthesizer (Biotage, Uppsala, Sweden). HPLC analyses were performed to determine the purity of the peptides. UV-light sensitive peptides were built with a 2-nitrophenylamino acid reside. Before usage peptides were solved in 10% DMSO, 0.5% TFA (VWR; #1.08262.0025) at a final concentration of 10 mg/mL and stored at −20° C. until further handling.

The UV-exchange of UV-light sensitive peptides was done as described in Rodenko et al.⁴⁵ The peptides were incubated for 1 h at 366 nm UV-light with a biotinylated UV-light sensitive pHLA complex at a molar ratio of 100:1. Obtained biotinylated UV-exchanged pHLA complexes were tetramerized with PE-coupled streptavidin (Invitrogen, #12-4317-87) in a 4:1 molar ratio. The calculated streptavidin-PE amount was added in three separate steps with a 30 min incubation period in the dark at 4° C. and 1500 rpm in a thermomixer C (Eppendorf, Hamburg, Germany). After that Biotin was added at a final concentration of 25 μM.

### Peptide Sequences

TABLE 1
Sequence information of similar peptides
Sequence at peptide position

| | | | | | | | | | | SEQ |
|---|---|---|---|---|---|---|---|---|---|---|
| | 1 | 2 | 3 | 4 | 5 | 6 | 7 | 8 | 9 | ID NO: |
| PRAME | S | L | L | Q | H | L | I | G | L | 015 |
| Similar 1 | S | . | L | Q | . | L | I | . | . | 016 |
| Similar 2 | S | L | . | . | H | L | . | G | L | 017 |
| Similar 3 | S | L | . | Q | H | L | . | . | . | 018 |
| Similar 4 | S | L | L | . | . | L | I | . | L | 019 |
| Similar 5 | S | L | L | . | H | L | . | . | L | 020 |
| Similar 6 | S | L | L | Q | H | . | L | . | L | 021 |
| Similar 7 | S | L | L | Q | . | | | . | L | 022 |
| Similar 8 | S | L | . | Q | . | L | . | G | . | 023 |
| Similar 9 | S | . | L | . | . | L | I | G | . | 024 |
| Similar 10 | . | L | L | . | H | . | I | G | L | 025 |
| Similar 11 | . | . | . | . | H | L | I | . | . | 026 |

“.” representing a variation to the PRAME peptide

### Biolayer Interferometry

An Octet HTX system (Sartorius, Goettingen, Germany) was used for the affinity measurements of soluble TCER molecules towards their target pHLA complexes. Kinetic binding analysis was performed using kinetics buffer (DPBS, 0.1% BSA, and 0.05% Tween-20 (Sigma Aldrich, #P1379-100ML)) to dilute all analytes to their final concentrations. Samples were loaded and measured in a 384 tilted well plate containing 60 μL sample volume at a 3 mm sensor off-set. The used HIS1K biosensors were hydrated with kinetics buffer for at least 10 min. For the measurements the plate was kept at a temperature of 30° C. and shaked at 1000 rpm. Baselines before association phases and the following dissociation phases were performed in the same wells to enable inter-step correction. All obtained data was analyzed using the Data Analysis HT software (version 12.0). Alignment of the raw sensor data at the Y axis was performed by adjusting the data to the end of the baseline step. Inter-step correction was applied for the alignment of the dissociation phase start to the end of the association phase. Savitzky-Golay filtering was applied to smooth data. Finally, fitting of the sensograms to a 1:1 Langmuir kinetics binding model was done.

### Data Analysis

Flow cytometry data were analyzed using the FlowJo 10.7 software (Becton Dickinson, Franklin Lake, USA).

Statistical analysis and data plotting was performed via GraphPad Prism 9.2.0 (GraphPad Prism Software, San Diego, USA). EC₅₀ values were derived from four parameter logistic sigmoidal non-linear regression.

Sequence analysis and construct planning was done within Geneious Prime software 2020.2.5 (Biomatters Ltd, Auckland, New Zealand).

### Example 1: Generation of High Expression CHO Cells for Targeted Protein Expression

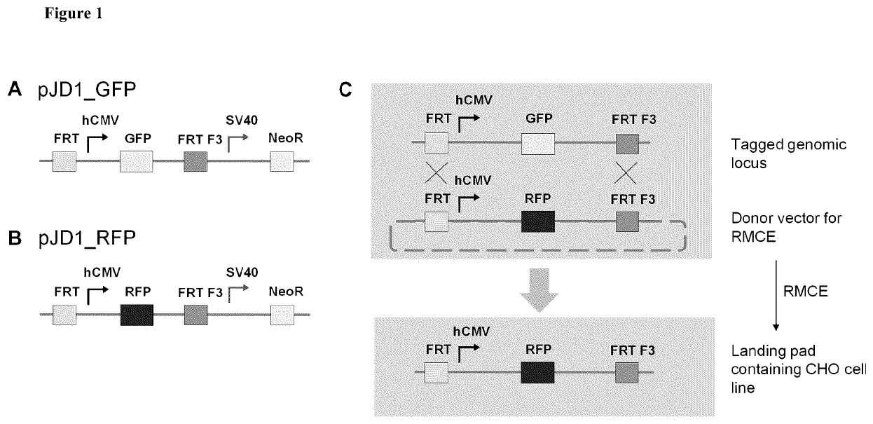

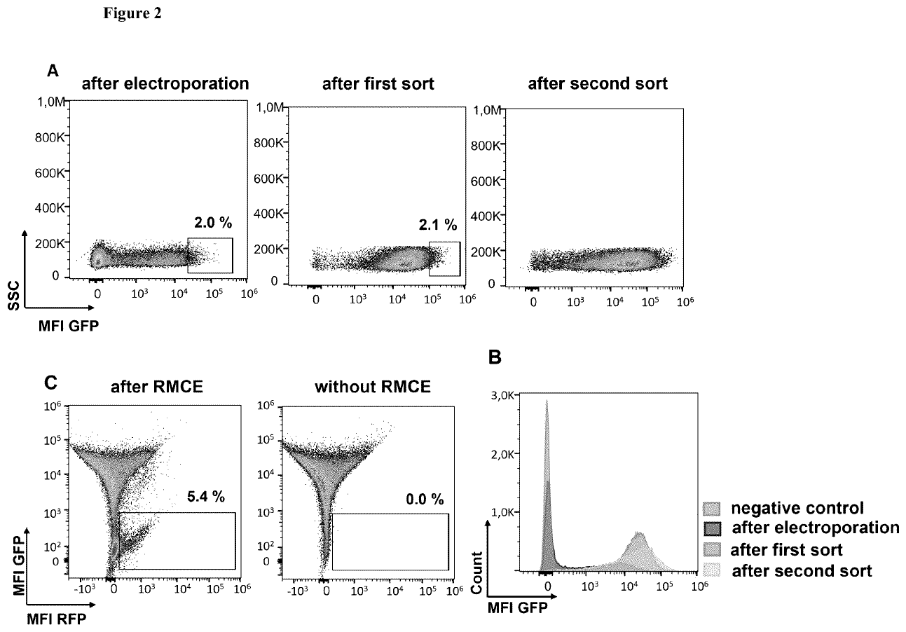

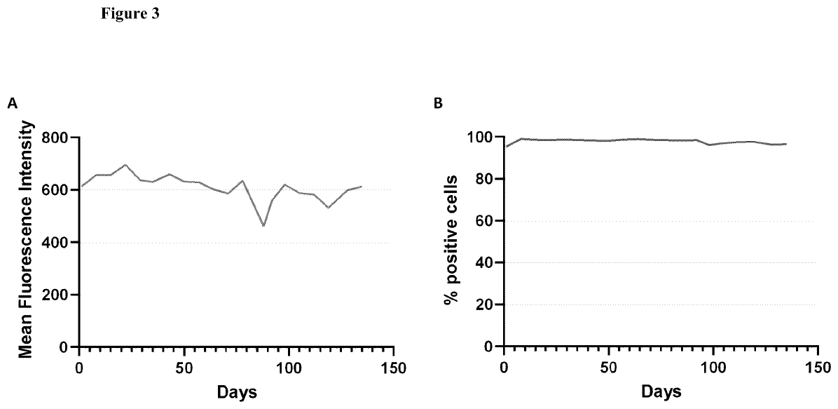

For the generation of a stable, high expression CHO cell line with a recombinase-mediated cassette exchange (RMCE)-based re-targetable landing pad, two vectors encoding for neomycin resistance and FRT-flanked fluorescence markers GFP or RFP were designed (FIG. 1). After electroporation of the GFP encoding vector pJD1_GFP into CHO cells and a subsequent culturing period of 14 days under Geneticin selection, about 2% of the cells with highest GFP expression were sorted by flow cytometry and the procedure was repeated including a second sort (FIG. 2). The resulting CHO cells showed stable and high expression of GFP caused by a random integration of the GFP cassette into the genome. The GFP expressing cells were then electroporated with the RFP encoding vector pJD1_RFP (FIG. 1 C) together with RNA encoding Flp recombinase to mediate RMCE. After the RMCE process, 5.4% of the CHO cells showed RFP expression in absence of any GFP expression supporting a high exchange rate of the RFP cassette (FIG. 2 C). In the next step, the RFP-positive/GFP-negative cells were subjected to single cell sort and CHO clones were generated for assessing the genomic integration site of the RFP cassette by targeted locus amplification analysis. The analysis revealed a CHO clone (RFP_A03) with a single copy integration of the RFP exchange cassette. To confirm stable, long-term expression of RFP the CHO clone, RFP_A03, was cultured for a period of 135 days in the absence of any Geneticin selection. During the long-term culture, RFP_A03 showed very stable and high expression level of the RFP fluorescence marker (FIG. 3) supporting the use of this clone for targeted protein expression. Therefore, the clone RFP_A03 was expanded as the stable, landing pad-containing cell line allowing targeted expression and engineering of proteins in CHO display.

### Example 2: Generation of PRAME-Specific TCER Library and Selection of Candidates

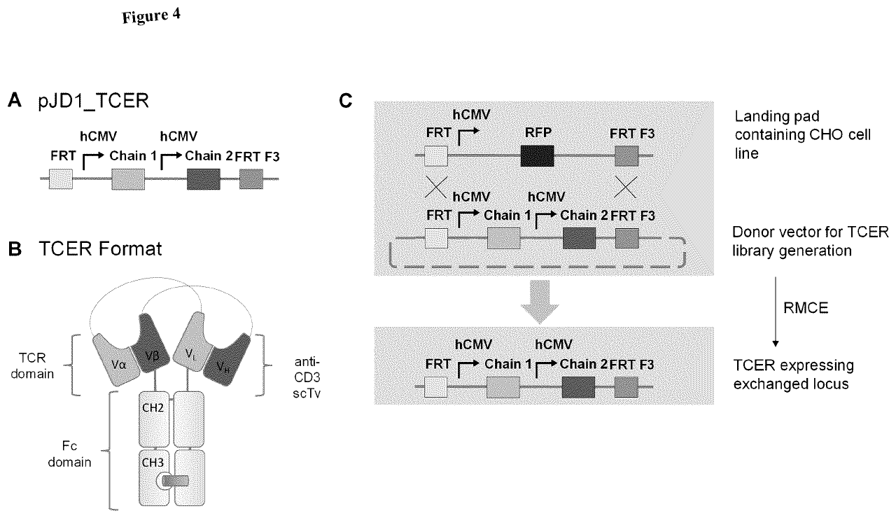

To demonstrate that RFP-A03 CHO cells can be used for displaying and engineering of complex biomolecules such as bispecific TCR molecules, a library of TCER molecules for targeted integration and expression was generated (FIG. 4). The TCER library was based on a PRAME-specific model TCR that was previously maturated by yeast display to increase stability and binding affinity and resulted in the identification of various variants of CDRα1, CDRα2, CDRα3, CDRβ1, CDRβ2, and CDRβ3 of the model TCR (Table 1). The individual CDR sequences of the best performing variants of the model TCR were employed to generate a library of TCER molecules for displaying on RFP_A03 CHO cells for identifying optimal combinations of the CDR variants. The TCER library design allowed the expression of TCER molecules covering every potential combination of the individual CDRs corresponding to a minimum library size of 36,864 TCER molecules derived from 2, 6, 16, 6, 16, and 2 variants of CDRα1, CDRα2, CDRα3, CDRβ1, CDRβ2, and CDRβ3, respectively (Table 1).

TABLE 2
CDR sequences for the combinatorial screening library in CHO cells.

| CDRα1 | CDRα2 | CDRα3 | CDRβ1 | CDRβ2 | CDRβ3 |
|---|---|---|---|---|---|
| DRGSQS | YSNGDKE | DNAHGGM | SGHRS | EHGLER | CASSPWDSPNVQY |
| (SID: 027) | (SID: 028) | (SID: 029) | (SID: | (SID: 031) | (SID: 032) |
| | | | 030) | | |
| DRGSQL | YQEGDKE | DNDQGGI | EGHRA | FSETQR | CASSPWDSPNEQY |
| (SID: 033) | (SID: 034) | (SID: 035) | (SID: | (SID: 037) | (SID: 038) |
| | | | 036) | | |
| | YQTGDKE | DNDVGGI | PGHKA | IHGEER | |
| | (SID: 039) | (SID: 040) | (SID: | (SID: 042) | |
| | | | 041) | | |
| | YQAGDKE | DNEQGGM | PGHRA | IHGQER | |
| | (SID: 043) | (SID: 044) | (SID: | (SID: 046) | |
| | | | 045) | | |
| | YPQGDKK | DNKAGGI | PGHRS | IHGVER | |
| | (SID: 047) | (SID: 048) | (SID: | (SID: 050) | |
| | | | 049) | | |
| | YSQGDKE | DNPAGGI | QGHRA | VHGAER | |
| | (SID: 051) | (SID: 052) | (SID: | (SID: 054) | |
| | | | 053) | | |
| | | DNPRGGM | | VHGEER | |
| | | (SID: 055) | | (SID: 056) | |
| | | DNPVGGP | | VHGIER | |
| | | (SID: 057) | | (SID: 058) | |
| | | ENKPGGP | | VHGKER | |
| | | (SID: 059) | | (SID: 060) | |
| | | GNAQGGM | | VHGLER | |
| | | (SID: 061) | | (SID: 062) | |
| | | GNDLGGI | | VHGMER | |
| | | (SID: 063) | | (SID: 064) | |
| | | NNPSGGM | | VHGNER | |
| | | (SID: 065) | | (SID: 066) | |
| | | PNPPGGK | | VHGQER | |
| | | (SID: 067) | | (SID: 068) | |
| | | PNTHGGP | | VHGRER | |
| | | (SID: 069) | | (SID: 070) | |
| | | SNFGNEK | | VHGVER | |
| | | (SID: 071) | | (SID: 072) | |
| | | TNIAGGS | | VHGYAR | |
| | | (SID: 073) | | (SID: 074) | |

SEQ ID NO (SID) indicated in brackets

TCER molecules showing strong and specific binding to the PRAME pHLA target were selected during three rounds of CHO cell sorting according to selection and counterselection principles. The first sorting round was done with 100 nM PRAME pHLA monomer without counterselection. In a second and a third sorting round 31.6 nM and 10 nM PRAME pHLA monomer, respectively, were used for TCER selection together with a counterselection based on 10 nM pHLA tetramers representing each of 11 different peptides being expressed on human normal tissues and showing a high degree of sequence similarity to the PRAME target peptide. From the TCER-displaying CHO cells sorted in the third round, 171 clones were sequenced resulting in 39 unique variable domain sequences of the model PRAME TCR. Based on the highest abundance of sequences and the highest strength of PRAME pHLA binding the inventors selected 10 TCER candidates for detailed characterization. Among the 10 selected TCER candidates, between 3 and 5 out of the 6 wild type (wt) CDRs appeared to be modified (Table 2). The most stringent effect was observed for CDR32 for which only 1 out of 16 input CDR variants (VHGEER) was identified indicating that this CDR32 variant had a major role for improving the TCER binding to PRAME pHLA. For CDRα2, CDRα3, CDRβ1, and CDRβ3, respectively, 8, 9, 7, and 5 variants were found among the 10 selected TCER candidates. However, given that the occurrence of variants for these 4 CDRs cannot clearly be distinguished from a random distribution, the contribution of these CDR variants to an improved affinity can hardly be determined. For CDRα1, no variant beyond the wt was identified arguing against a contribution of this CDR in increasing TCR affinity.

TABLE 3
CDR sequences of selected TCER candidates from the CDR combinatorial screening in
comparison to the wild type CDRs of the parental TCR.

| Clone | CDRα1 | CDRα2 | CDRα3 | CDRβ1 | CDRβ2 | CDRβ3 |
|---|---|---|---|---|---|---|
| parental | DRGSQS | YSNGDKE | SNFGNEK | SGHRS | FSETGR | CASSPWDS |
| TCR | (SID: 075) | (SID: 076) | (SID: 077) | (SID: 078) | (SID: 079) | PNEQY |
| | | | | | | (SID: 080) |
| CL-7435 | DRGSQS | YQAGDKE | GNDLGGI | SGHRS | VHGEER | CASSPWDS |
| | (SID: 081) | (SID: 082) | (SID: 083) | (SID: 084) | (SID: 085) | PNEQY |
| | | | | | | (SID: 086) |
| CL-7445 | DRGSQS | YSNGDKE | DNPRGGM | QGHRA | VHGEER | CASSPWDS |
| | (SID: 087) | (SID: 088) | (SID: 089) | (SID: 090) | (SID: 091) | PNEQY |
| | | | | | | (SID: 092) |
| CL-7467 | DRGSQS | YQAGDKE | GNAQGGM | PGHRA | VHGEER | CASSPWDS |
| | (SID: 093) | (SID: 094) | (SID: 095) | (SID: 096) | (SID: 097) | PNVQY |
| | | | | | | (SID: 098) |
| CL-7475 | DRGSQS | YPQGDKK | DNPAGGI | SGHRS | VHGEER | CASSPWDS |
| | (SID: 099) | (SID: 100) | (SID: 101) | (SID: 102) | (SID: 103) | PNVQY |
| | | | | | | (SID: 104) |
| CL-7480 | DRGSQS | YQEGDKE | SNFGNEK | PGHRA | VHGEER | CASSPWDS |
| | (SID: 105) | (SID: 106) | (SID: 107) | (SID: 108) | (SID: 109) | PNEQY |
| | | | | | | (SID: 110) |
| CL-11581 | DRGSQS | YSQGDKE | DNPRGGM | PGHRS | VHGEER | CASSPWDS |
| | (SID: 111) | (SID: 112) | (SID: 113) | (SID: 114) | (SID: 115) | PNVQY |
| | | | | | | (SID: 116) |
| CL-11594 | DRGSQS | YSNGDKE | DNEQGGM | PGHRS | VHGEER | CASSPWDS |
| | (SID: 117) | (SID: 118) | (SID: 119) | (SID: 120) | (SID: 121) | PNVQY |
| | | | | | | (SID: 122) |
| CL-11611 | DRGSQS | YQEGDKE | NNPSGGM | QGHRA | VHGEER | CASSPWDS |
| | (SID: 123) | (SID: 124) | (SID: 125) | (SID: 126) | (SID: 127) | PNEQY |
| | | | | | | (SID: 128) |
| CL-11614 | DRGSQS | YSQGDKE | DNPAGGI | SGHRS | VHGEER | CASSPWDS |
| | (SID: 129) | (SID: 130) | (SID: 131) | (SID: 132) | (SID: 133) | PNEQY |
| | | | | | | (SID: 134) |
| CL-11623 | DRGSQS | YSQGDKE | NNPSGGM | PGHRS | VHGEER | CASSPWDS |
| | (SID: 135) | (SID: 136) | (SID: 137) | (SID: 138) | (SID: 139) | PNVQY |
| | | | | | | (SID: 140) |

SEQ ID NO (SID) indicated in brackets

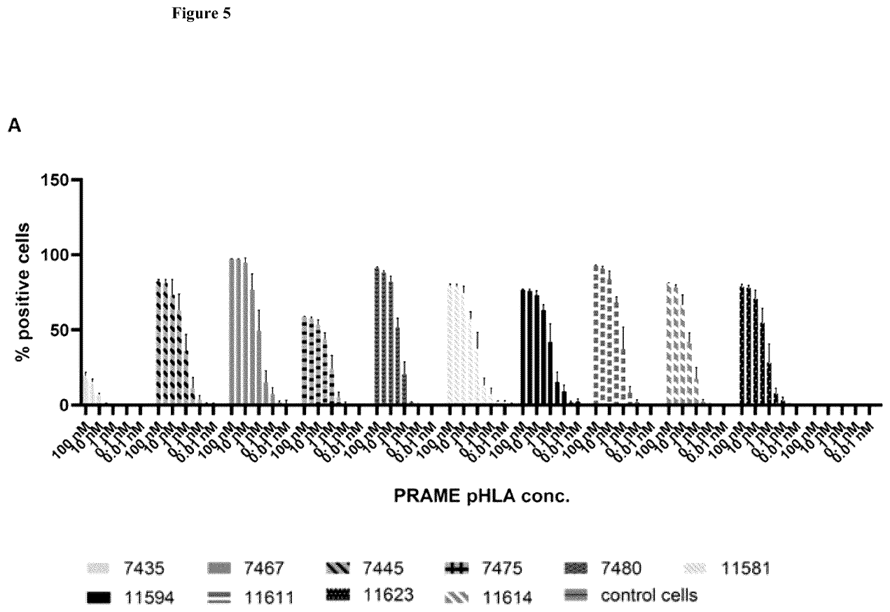

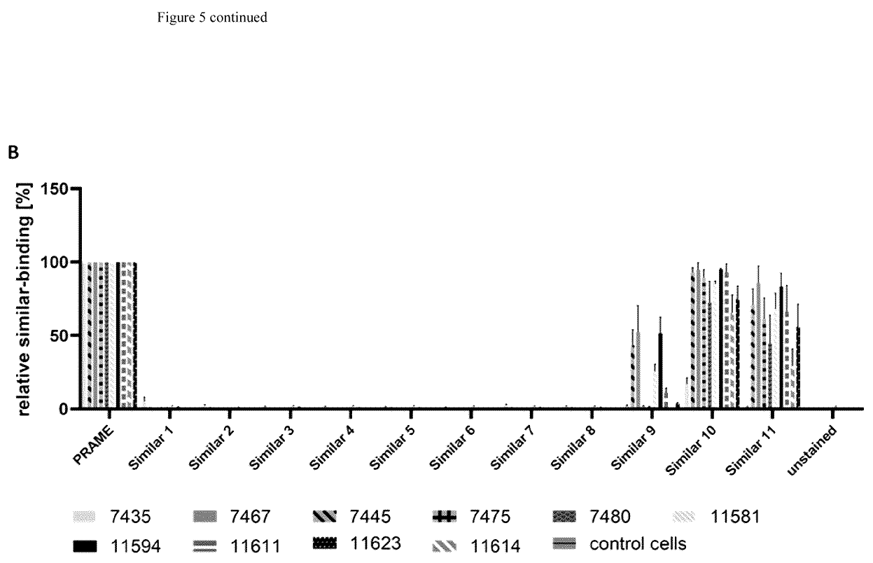

The selected TCER candidates (Table 2) were further assessed regarding their binding specificity profile. With exception of the weaker binding candidates CL-7435, CL-7475, CL-7480, and CL-11614, the TCER candidates showed strong binding to PRAME pHLA monomers with first detectable binding signals at 100 pM monomer concentration (FIG. 5A). Next the binding specificity of the TCER candidates was analyzed using a highly sensitive, avidity-driven analysis with pHLA tetramers each constituting one of 11 different peptides with a high degree of sequence similarity to the PRAME target peptide. As shown in FIG. 51B, the TCER candidates showed moderate to strong binding to 3 out of the 11 tested similar peptides. Strongest binding was observed for similar peptide 10 whose sequence is identical to the PRAME target peptide in 6 out of 9 positions (Table 1). The weakest binding was observed towards similar peptide 9 sharing 5 identical positions with the PRAME target peptide. Interestingly, moderate to high binding was also seen with similar peptide 11 sharing only the identical positions 5, 6, and 7 with the PRAME target peptide arguing for a relevant role of the c-terminal peptide stretch for binding of the TCER candidates.

### Example 3: Assessment of TCER-Mediated Anti-Tumor Activity

For functional testing, all selected TCER candidates (Table 2) were expressed in CHO cells and the purified molecules were subjected to affinity measurements using biolayer interferometry. As shown in Table 3, TCER candidates CL-7467 and CL-7445 exhibited the highest PRAME pHLA binding affinity with a KD of 3.4 and 3.7 nM, respectively. Lowest binding affinities of KD of 16.5, 17.8, 24.5, and 37.4 nM were determined for the candidates CL-7435, CL-11614, CL-7480, and CL-7475, respectively, which is in line with the lack of binding signals of these candidates observed in CHO display when only 0.1 nM PRAME pHLA were used for the staining (see also FIG. 5A).

TABLE 4
PRAME pHLA binding affinity of selected soluble TCER
candidates in comparison to the wild type CDR-harboring
parental TCR expressed as single chain TCR

| | K_{D} |
|---|---|
| Clone | [nM] |
| CL-7467 | 3.4 |
| CL-7445 | 3.7 |
| CL-11581 | 5.2 |
| CL-11594 | 6.1 |
| CL-11623 | 6.6 |
| CL-11611 | 12.0 |
| CL-7435 | 16.5 |
| CL-11614 | 17.8 |
| CL-7480 | 24.5 |
| CL-7475 | 37.4 |
| Parental TCR | 1230.0 |

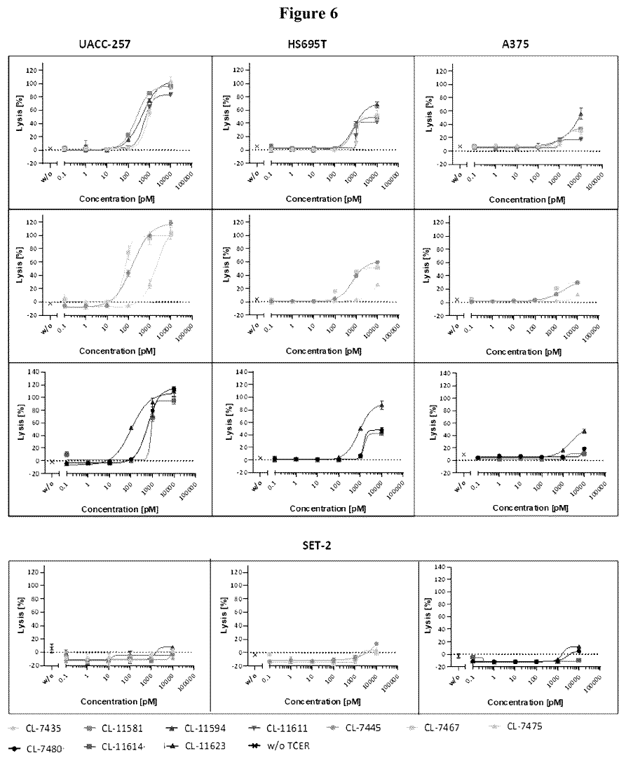

In the next step, the ability of the selected TCER candidates to induce PRAME peptide dependent tumor cell lysis was assessed. LDH-release assays were performed with the target cell lines UACC-257, HS695T, and A375 presenting the PRAME pHLA at different target densities. The PRAME peptide copy numbers per cell (cpc) ranged from about 1100 cpc for UACC-257, 400-550 cpc for HS695T, and about 50 cpc for A375. As control, SET-2 cells were included in the experiments, which had no detectable PRAME peptide copies on their surface. All selected TCER candidates induced target cell killing of UACC-257 and HS695T cells (FIG. 6) albeit lowest reactivity was seen with the candidates CL-7475, CL-7480, and CL-11614 in line with their lower binding affinity (Table 4). For A375 cells presenting the target PRAME peptide only at a very low density (50 cpc), weak killing activity was detectable only for TCER candidates showing higher affinity such as CL-7467, CL-7445, CL-11581, CL 11594, and CL-11623. In line with the expectation, there was none or only very weak killing activity at the highest TCER concentration observed for all tested candidates with PRAME-negative SET-2 cells. Table 5 summarizes the EC50 values calculated from the LDH-release assays with UACC-257 and HS695T cells arguing for CL-7467 as the TCER candidate with the highest anti tumor activity in line with its highest binding affinity.

TABLE 5
EC₅₀ values based on cytotoxicity data from selected candidates.

| | EC₅₀ | EC₅₀ |
|---|---|---|
| | UACC-257 | HS695T |
| Name | [pM] | [pM] |
| CL-7435 | 1136 | 1380 |
| CL-7445 | 115 | 644 |
| CL-7467 | 61 | 172 |
| CL-7475 | 1864 | 1556 |
| CL-7480 | 730 | 1849 |
| CL-11581 | 310 | 587 |
| CL-11594 | 450 | 808 |
| CL-11611 | 656 | 984 |
| CL-11614 | 914 | 1424 |
| CL-11623 | 136 | 849 |

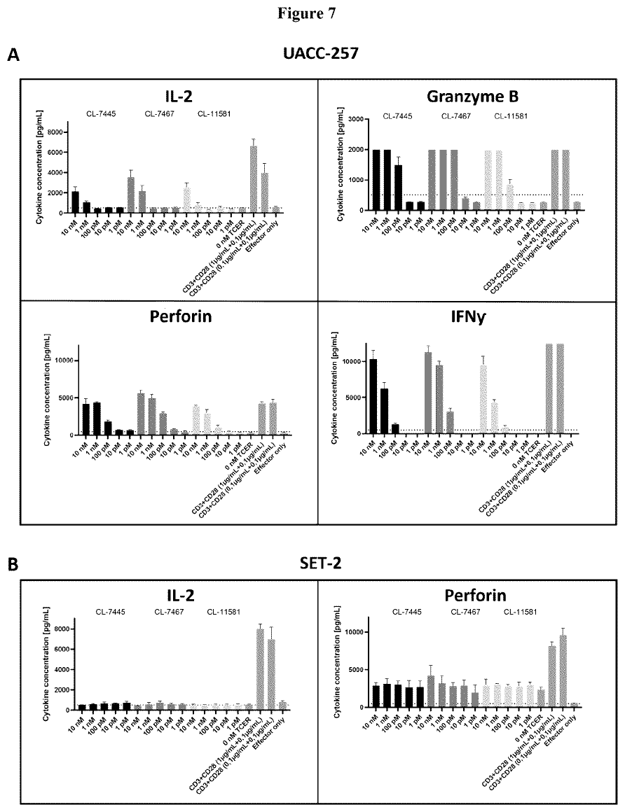

The inventors also determined cytokine levels from the killing assay co-cultures to get more insight into the immune reactions triggered by the three potent TCER candidates CL-7445, CL-7467, and CL-11581. As shown in FIG. 7, the tested candidates induced release of IL-2, granzyme B, perforin, and IFNγ from human PBMC in response to UACC 257 tumor cells. Among the three candidates, CL-7467 showed highest capacity in inducing above-mentioned cytokines with pronounced release of perforin, granzyme B, and IFNγ already at 100 pM TCER concentration while IL-2 release was pronounced at 1 nM. As expected, no induction of IL-2 and perforin release was observed for all three TCER candidates when PBMC were co-cultured with the PRAME-negative cell line SET-2.

### Conclusions on the Exemplary Use of the Invention

The present invention discloses herein above the exemplary setup and application of a mammalian display platform for targeted expression and engineering of therapeutic proteins, which teaches in a general fashion also different related uses as explained throughout the text of this specification.

In particular, the exemplary platform disclosed herein employs CHO cells genetically modified to contain a landing pad allowing for RCME-mediated integration of genetic elements encoding for biomolecules. Genetic information integrated into CHO cells by this process will be expressed from a single copy at the same genetic locus. Such a targeted single copy integration has significant advantages over display systems based on standard transfection, viral transduction, and transposases, which often result in incorporation of multiple copies at different integration sites (Beerli et al., Proc National Acad Sci 2008; 105:14336-41; Breous-Nystrom et al., Methods 2014; 65:57-67; Waldmeier et al., Mabs 2016; 8:726-40) leading to variations in the protein expression levels making subsequent screening and selection processes complicated (Valldorf et al., Biol Chem 2021; 0:000010151520200377). To facilitate the exchange efficiency, the inventors applied an electroporation protocol in which RNA encoded Flp recombinase together with the DNA encoded donor vector was used for CHO cell transfection. This resulted in exchange rates of about 5% and compares favorably with reported exchange efficiencies achieved with alternative technologies designed for targeted protein engineering like CRISPR/Cas9 and TALEN showing also up to 5% exchange efficiency (Parthiban et al., Mabs 2019; 11:884-98; Parola et al., Mabs 2019; 11:1367-80). In addition, by using an enhanced Flp version, the exchange efficiency rates may even be increased to more than 7% (Phan et al., Sci Rep-uk 2017; 7:17771). Thus, in combination with a MaxCyte flow electroporation system that enables electroporation of up to 1×10¹¹ cells in a 30-min cycle, the above exchange rates would allow generation of large mammalian display libraries with a theoretical size of about 1×10¹ (Parthiban et al., Mabs 2019; 11:884-98). In the herein discloses exemplary study the inventors worked with a small library of 10⁴-10⁵ with the main goal to demonstrate usage of the generated CHO display system for engineering TCR domains in context of the full-length, bispecific TCER format. Although TCR variable domains have been affinity maturated by means of other display methods, such as phage display and yeast display, to the best of the inventor's knowledge this is the first demonstration of feasibility of TCR engineering in the context of a final bispecific ABP. Since CHO cells tend to express mutations identified from a CHO-based system better than mutations found in other host cells (Chen et al., Biotechnol Bioeng 2016; 113:39-51), the use of CHO cells for engineering of multispecific ABPs may have benefits for later production of the molecules in CHO cells. In addition, the usage of the final bispecific TCER format makes subsequent reformatting into the therapeutic format, which can be accompanied by loss of affinity or even loss of function, dispensable (Steinwand et al., Mabs 2013; 6:204-18; Robertson; Robertson et al., J Biol Chem 2020; 295:18436-48).

Furthermore, maturation of the TCR domains in the final bispecific format can potentially also identify beneficial mutations resulting in an increased interdomain stability between TCR and antibody variable domains of the TCER format, an aspect that is not possible with a reformatting process. Based on the measurement of RFP fluorescence intensity the inventors could confirm a highly stable expression of the inserted RFP cassette in CHO cells for a period of 135 days. This long-term expression stability in CHO cells outperforms other expression systems based on episomal vectors or random integration (Bowers et al., Proc National Acad Sci 2011; 108:20455-60; Chen et al., Protein Cell 2012; 3:460-9) and is more than sufficient to perform several rounds of biomolecule selection during an engineering campaign. For the disclosed engineering campaign the inventors selected a model TCR targeting an HLA-A*02-presented peptide derived from PRAME. The parental PRAME model TCR displaying non-modified CDRs exhibited a binding affinity of 1.2 μM (KD) when expressed as single chain TCR construct that harbored four stabilizing TCR framework mutations for soluble expression. In a previous maturation campaign based on yeast surface display we selected binding improved CDR variants of the PRAME model TCR, which were further employed for the invention disclosed herein to identify an optimal combination of the different CDR variants. In this disclosure the inventors used the stabilized variable domain sequences and the different CDR variants of the PRAME model TCR to generate a TCER library in CHO cells allowing for a systematic combination of all identified CDR variants to select PRAME TCER candidates with improved binding characteristics. The inventors identified 39 unique PRAME TCR sequences each displaying different combinations of the input CDR sequences. Interestingly, among the best binding and most frequent TCER candidates, only one CDR32 was identified out of the 16 CDR32 input sequences of the library supporting a successful selection process in CHO display resulting in TCER variants with improved PRAME pHLA binding. The inventors selected 10 TCER candidates for detailed assessment of PRAME pHLA binding of CHO-displayed TCER in comparison to functional anti tumor cell responses mediated by their soluble counterparts expressed in CHO cells. In general, good correlation between PRAME pHLA binding of CHO-displayed TCER and its behavior as soluble bispecific was observed since the only four TCER candidates CL-7435, CL-7475, CL7480, and CL-11614 lacking a CHO display binding signal with 0.1 nM PRAME pHLA also showed the weakest binding affinity in biolayer interferometry and the lowest reactivity against PRAME pHLA positive tumor cells. This observation thus demonstrates the use of CHO display for predicting highly functional TCER candidates. The best TCER candidate identified in CHO display, CL-7467, exhibited highest binding affinity as measured with biolayer interferometry of 3.4 nM (Kd), which is an about 400-fold increase over the parental TCR variants, and also showed highest anti-tumor cell reactivity together with the most pronounced release of perforin, granzyme B, IFNγ, and IL-2.

A prerequisite for successful TCR-based therapeutics is the ability to provoke a strong, but at the same time highly specific anti-tumor response. Thus, the assessment of unpredicted cross-reactivity is of high importance to minimize potential safety risks during clinical development. This aspect is highlighted by a severe case of cross reactivity of an autologous TCR-T program using an affinity-enhanced TCR that targeted besides the intended MAGE-A3 target peptide also a similar peptide expressed in cardiomyocytes leading to fatalities in the clinical trial (Cameron et al., Sci Transl Med 2013; 5:197ra103; Linette et al., Blood 2013; 122:863-71). Using the herein disclosed mammalian cell display system, an unintended cross-reactivity of TCER molecules can be evaluated early in the development process by testing the TCER binding towards normal tissue presented peptides with high sequence similarity to the target peptide. The herein disclosed data demonstrate the high value of the established mammalian cell display system for maturation of multispecific TCR-based molecules and support their usage for the generation of pHLA targeting multispecific ABPs.

1. A method of providing a multispecific antigen binding protein (ABP), wherein the multispecific ABP is comprising at least one T-cell receptor (TCR)-derived binding site, comprising the steps of:
(I) generating a library of nucleic acids encoding multispecific ABPs, which comprises different variants of the multispecific ABPs;
(II) providing a multitude of mammalian cells each expressing one member of the library of step (I) on their cell surface;
(III) contacting the mammalian cells with a first target of the multispecific ABP and selecting a mammalian cell expressing a multispecific ABP binding to the first target;
(IV) optionally contacting the mammalian cell selected in step (III) with a further target of the multispecific ABP and selecting a mammalian cell expressing a multispecific ABP binding to the further target;
(V) isolating a multispecific ABP from the selected mammalian cell of step (III) or if performed step (IV).

2. The method according to claim 1, further comprising the step of:
(VI) determining at least one property of the isolated multispecific ABP selected from stability, solubility, production yield and hydrophobic patches.

3. The method according to claim 1, further comprising the steps
(IIIa) contacting the selected mammalian cell of step (III) with a variant of the first target and selecting a mammalian cell expressing a multispecific ABP binding to the variant of the first target with a lower binding affinity than binding to the first target;
(IVa) optionally contacting the selected mammalian cell of step (IV) with a variant of the further target and selecting a mammalian cell expressing a multispecific ABP binding to the variant of the further target with a lower binding affinity than binding to the further target.

4. The method according to claim 1, wherein the first target, and if applicable the further target, other target and variants thereof is a peptide, optionally an MHC-associated peptide epitope.

5. The method according to claim 1, wherein the multispecific ABP is comprising at least one antibody-derived binding site.

6. The method according to claim 1, wherein the multispecific ABP is a bispecific ABP.

7. The method according to claim 1, wherein the first target is an MHC associated peptide epitope and/or wherein the further target is a cell surface antigen of a human immune effector cell.

8. The method according to claim 1, wherein the multispecific ABP is a bispecific polypeptide molecule comprising a first polypeptide chain and a second polypeptide chain, wherein:
a) the first polypeptide chain comprises
ai) a first variable domain (VD1) of an antibody,
aii) a first variable domain (VR1) of a T cell receptor (TCR), and
aiii) a first linker (LINK1) connecting said domains;
b) the second polypeptide chain comprises
bi) a second variable domain (VR2) of a TCR,
bii) a second variable domain (VD2) of an antibody, and
biii) a second linker (LINK2) connecting said domains;
wherein the first variable domain (VR1) is one of a TCR Vα domain and a TCR Vβ domain, and the second variable domain (VR2) is the other one of the TCR Vα domain and the TCR Vβ domain, said first variable domain (VR1) and said second variable domain (VR2) associate to form a first binding site (VR1)(VR2) that specifically binds the first target, optionally an MHC-associated peptide epitope;
wherein said first variable domain (VD1) and said second variable domain (VD2) associate to form a second binding site (VD1)(VD2) that specifically binds the further target, optionally a cell surface antigen of a human immune effector cell;
wherein said two polypeptide chains are fused to human IgG hinge domains and/or human IgG Fc domains or dimerizing portions thereof,
wherein said two polypeptide chains are connected by covalent and/or non-covalent bonds between said hinge domains and/or Fc-domains;
wherein said dual specificity polypeptide molecule is capable of simultaneously binding the cell surface molecule and the MHC-associated peptide epitope; and
wherein the order of the variable domains in the two polypeptide chains is selected from VD1-VR1 and VR2-VD2 or VD2-VR2 and VR1-VD1.

9. The method according to claim 8, wherein the linker-sequences LINK1 and/or LINK2 having a length of 6 to 12 amino acids, optionally 7 to 9 amino acids, optionally 8 amino acids.

10. The method according to claim 1, wherein the multispecific ABP binds the first target of step (III) with a binding affinity of at least 1 pM to 100 nM, optionally 1 nM to 10 nM.

11. The method according to claim 1, wherein the multispecific ABP encoded in the library of step (I) is fused to a transmembrane domain, optionally a PDGFR transmembrane domain.

12. The method according to claim 3, wherein step (IIIa) is performed simultaneous with step (III) and/or step (IVa) is performed simultaneous with step (IV).

13. The method according to claim 3, wherein the variant of the first target and/or the variant of the further target(s):
(i) is expressed in healthy, optionally non-cancerous, tissue; and/or
(ii) is displayed as an MHC-associated peptide epitope.

14. The method according to claim 2, further comprising the steps
(IIIa) contacting the selected mammalian cell of step (III) with a variant of the first target and selecting a mammalian cell expressing a multispecific ABP binding to the variant of the first target with a lower binding affinity than binding to the first target;
(IVa) optionally contacting the selected mammalian cell of step (IV) with a variant of the further target and selecting a mammalian cell expressing a multispecific ABP binding to the variant of the further target with a lower binding affinity than binding to the further target.

15. The method according to claim 2, wherein the first target, and if applicable the further target, other target and variants thereof is a peptide, optionally an MHC-associated peptide epitope.

16. The method according to claim 3, wherein the first target, and if applicable the further target, other target and variants thereof is a peptide, optionally an MHC-associated peptide epitope.

17. The method according to claim 2, wherein the multispecific ABP is comprising at least one antibody-derived binding site.

18. The method according to claim 3, wherein the multispecific ABP is comprising at least one antibody-derived binding site.

19. The method according to claim 4, wherein the multispecific ABP is comprising at least one antibody-derived binding site.

20. The method according to claim 2, wherein the multispecific ABP is a bispecific ABP


SEQUENCE LISTING (141 sequences)

SEQ ID NO: 1 - protein, 21 aa, Homo sapiens
EPKSCDKTHTCPPCPAPELLG

SEQ ID NO: 2 - protein, 19 aa, Homo sapiens
ERKCCVECPPCPAPPVAGP

SEQ ID NO: 3 - protein, 53 aa, Homo sapiens
ELKTPLGDTTHTCPRCPEPKSCDTPPPCPRCPEPKSCDTPPPCPRCPAPELLG

SEQ ID NO: 4 - protein, 18 aa, Homo sapiens
ESKYGPPCPSCPAPEFLG

SEQ ID NO: 5 - protein, 4 aa, synthetic construct
  REGION 1..4: note = Artificial Sequence
GGGS

SEQ ID NO: 6 - protein, 5 aa, synthetic construct
  REGION 1..5: note = 006
GGGGS

SEQ ID NO: 7 - protein, 5 aa, synthetic construct
  REGION 1..5: note = Artificial sequence
TVLRT

SEQ ID NO: 8 - protein, 6 aa, synthetic construct
  REGION 1..6: note = Artificial sequence
TVSSAS

SEQ ID NO: 9 - protein, 7 aa, synthetic construct
  REGION 1..7: note = Artificial sequence
TVLSSAS

SEQ ID NO: 10 - protein, 8 aa, synthetic construct
  REGION 1..8: note = Artificial sequence
GGGSGGGG

SEQ ID NO: 11 - DNA, 17 bp, synthetic construct
  misc_feature 1..17: note = primer JD061
  source 1..17: mol_type = other DNA
gacacgaagctggctag

SEQ ID NO: 12 - DNA, 18 bp, synthetic construct
  misc_feature 1..18: note = Primer JD065
  source 1..18: mol_type = other DNA
cttgctggcagaagtagg

SEQ ID NO: 13 - DNA, 17 bp, synthetic construct
  misc_feature 1..17: note = Primer JD057
  source 1..17: mol_type = other DNA
cagcgcctactctgagg

SEQ ID NO: 14 - DNA, 21 bp, synthetic construct
  misc_feature 1..21: note = Primer JD059
  source 1..21: mol_type = other DNA
gctggatatctgcagaattcc

SEQ ID NO: 15 - protein, 9 aa, synthetic construct
  REGION 1..9: note = PRAME
SLLQHLIGL

SEQ ID NO: 16 - protein, 9 aa, synthetic construct
  VARIANT 1..9: note = Similar 1
  VARIANT 2: note = Xaa can be any naturally occurring amino acid
  VARIANT 5: note = Xaa can be any naturally occurring amino acid
  VARIANT 8..9: note = Xaa can be any naturally occurring amino acid
SXLQXLIXX

SEQ ID NO: 17 - protein, 9 aa, synthetic construct
  VARIANT 1..9: note = Similar 2
  VARIANT 3..4: note = Xaa can be any naturally occurring amino acid
  VARIANT 7: note = Xaa can be any naturally occurring amino acid
SLXXHLXGL

SEQ ID NO: 18 - protein, 9 aa, synthetic construct
  VARIANT 1..9: note = Similar 3
  VARIANT 3: note = Xaa can be any naturally occurring amino acid
  VARIANT 7..9: note = Xaa can be any naturally occurring amino acid
SLXQHLXXX

SEQ ID NO: 19 - protein, 9 aa, synthetic construct
  VARIANT 1..9: note = Similar 4
  VARIANT 4..5: note = Xaa can be any naturally occurring amino acid
  VARIANT 8: note = Xaa can be any naturally occurring amino acid
SLLXXLIXL

SEQ ID NO: 20 - protein, 9 aa, synthetic construct
  VARIANT 1..9: note = Similar 5
  VARIANT 4: note = Xaa can be any naturally occurring amino acid
  VARIANT 7..8: note = Xaa can be any naturally occurring amino acid
SLLXHLXXL

SEQ ID NO: 21 - protein, 9 aa, synthetic construct
  VARIANT 1..9: note = Similar 6
  VARIANT 6: note = Xaa can be any naturally occurring amino acid
  VARIANT 8: note = Xaa can be any naturally occurring amino acid
SLLQHXLXL

SEQ ID NO: 22 - protein, 9 aa, synthetic construct
  VARIANT 1..9: note = Similar 7
  VARIANT 5..8: note = Xaa can be any naturally occurring amino acid
SLLQXXXXL

SEQ ID NO: 23 - protein, 9 aa, synthetic construct
  VARIANT 1..9: note = Similar 8
  VARIANT 3: note = Xaa can be any naturally occurring amino acid
  VARIANT 5: note = Xaa can be any naturally occurring amino acid
  VARIANT 7: note = Xaa can be any naturally occurring amino acid
  VARIANT 9: note = Xaa can be any naturally occurring amino acid
SLXQXLXGX

SEQ ID NO: 24 - protein, 9 aa, synthetic construct
  VARIANT 1..9: note = Similar 9
  VARIANT 2: note = Xaa can be any naturally occurring amino acid
  VARIANT 4..5: note = Xaa can be any naturally occurring amino acid
  VARIANT 9: note = Xaa can be any naturally occurring amino acid
SXLXXLIGX

SEQ ID NO: 25 - protein, 9 aa, synthetic construct
  VARIANT 1..9: note = Similar 10
  VARIANT 1: note = Xaa can be any naturally occurring amino acid
  VARIANT 4: note = Xaa can be any naturally occurring amino acid
  VARIANT 6: note = Xaa can be any naturally occurring amino acid
XLLXHXIGL

SEQ ID NO: 26 - skipped

SEQ ID NO: 27 - protein, 6 aa, synthetic construct
  REGION 1..6: note = CDRalpha1
DRGSQS

SEQ ID NO: 28 - protein, 7 aa, synthetic construct
  REGION 1..7: note = Artificial sequence
YSNGDKE

SEQ ID NO: 29 - protein, 7 aa, synthetic construct
  REGION 1..7: note = Artificial sequence
DNAHGGM

SEQ ID NO: 30 - protein, 5 aa, synthetic construct
  REGION 1..5: note = Artificial sequence
SGHRS

SEQ ID NO: 31 - protein, 6 aa, synthetic construct
  REGION 1..6: note = Artificial sequence
EHGLER

SEQ ID NO: 32 - protein, 13 aa, synthetic construct
  REGION 1..13: note = Artificial sequence
CASSPWDSPNVQY

SEQ ID NO: 33 - protein, 6 aa, synthetic construct
  REGION 1..6: note = Artificial sequence
DRGSQL

SEQ ID NO: 34 - protein, 7 aa, synthetic construct
  REGION 1..7: note = Artificial sequence
YQEGDKE

SEQ ID NO: 35 - protein, 7 aa, synthetic construct
  REGION 1..7: note = Artificial sequence
DNDQGGI

SEQ ID NO: 36 - protein, 5 aa, synthetic construct
  REGION 1..5: note = Artificial sequence
EGHRA

SEQ ID NO: 37 - protein, 6 aa, synthetic construct
  REGION 1..6: note = Artificial sequence
FSETQR

SEQ ID NO: 38 - protein, 13 aa, synthetic construct
  REGION 1..13: note = Artificial sequence
CASSPWDSPNEQY

SEQ ID NO: 39 - protein, 7 aa, synthetic construct
  REGION 1..7: note = Artificial sequence
YQTGDKE

SEQ ID NO: 40 - protein, 7 aa, synthetic construct
  REGION 1..7: note = Artificial sequence
DNDVGGI

SEQ ID NO: 41 - protein, 5 aa, synthetic construct
  REGION 1..5: note = Artificial sequence
PGHKA

SEQ ID NO: 42 - protein, 6 aa, synthetic construct
  REGION 1..6: note = Artificial sequence
IHGEER

SEQ ID NO: 43 - protein, 7 aa, synthetic construct
  REGION 1..7: note = Artificial sequence
YQAGDKE

SEQ ID NO: 44 - protein, 7 aa, synthetic construct
  REGION 1..7: note = Artificial sequence
DNEQGGM

SEQ ID NO: 45 - protein, 5 aa, synthetic construct
  REGION 1..5: note = Artificial sequence
PGHRA

SEQ ID NO: 46 - protein, 6 aa, synthetic construct
  REGION 1..6: note = Artificial sequence
IHGQER

SEQ ID NO: 47 - protein, 7 aa, synthetic construct
  REGION 1..7: note = Artificial sequence
YPQGDKK

SEQ ID NO: 48 - protein, 7 aa, synthetic construct
  REGION 1..7: note = Artificial sequence
DNKAGGI

SEQ ID NO: 49 - protein, 5 aa, synthetic construct
  REGION 1..5: note = Artificial sequence
PGHRS

SEQ ID NO: 50 - protein, 6 aa, synthetic construct
  REGION 1..6: note = Artificial sequence
IHGVER

SEQ ID NO: 51 - protein, 7 aa, synthetic construct
  REGION 1..7: note = Artificial sequence
YSQGDKE

SEQ ID NO: 52 - protein, 7 aa, synthetic construct
  REGION 1..7: note = Artificial sequence
DNPAGGI

SEQ ID NO: 53 - protein, 5 aa, synthetic construct
  REGION 1..5: note = Artificial sequence
QGHRA

SEQ ID NO: 54 - protein, 6 aa, synthetic construct
  REGION 1..6: note = Artificial sequence
VHGAER

SEQ ID NO: 55 - protein, 7 aa, synthetic construct
  REGION 1..7: note = Artificial sequence
DNPRGGM

SEQ ID NO: 56 - protein, 6 aa, synthetic construct
  REGION 1..6: note = Artificial sequence
VHGEER

SEQ ID NO: 57 - protein, 7 aa, synthetic construct
  REGION 1..7: note = Artificial sequence
DNPVGGP

SEQ ID NO: 58 - protein, 6 aa, synthetic construct
  REGION 1..6: note = Artificial sequence
VHGIER

SEQ ID NO: 59 - protein, 7 aa, synthetic construct
  REGION 1..7: note = Artificial sequence
ENKPGGP

SEQ ID NO: 60 - protein, 6 aa, synthetic construct
  REGION 1..6: note = Artificial sequence
VHGKER

SEQ ID NO: 61 - protein, 7 aa, synthetic construct
  REGION 1..7: note = Artificial sequence
GNAQGGM

SEQ ID NO: 62 - protein, 6 aa, synthetic construct
  REGION 1..6: note = Artificial sequence
VHGLER

SEQ ID NO: 63 - protein, 7 aa, synthetic construct
  REGION 1..7: note = Artificial sequence
GNDLGGI

SEQ ID NO: 64 - protein, 6 aa, synthetic construct
  REGION 1..6: note = Artificial sequence
VHGMER

SEQ ID NO: 65 - protein, 7 aa, synthetic construct
  REGION 1..7: note = Artificial sequence
NNPSGGM

SEQ ID NO: 66 - protein, 6 aa, synthetic construct
  REGION 1..6: note = Artificial sequence
VHGNER

SEQ ID NO: 67 - protein, 7 aa, synthetic construct
  REGION 1..7: note = Artificial sequence
PNPPGGK

SEQ ID NO: 68 - protein, 6 aa, synthetic construct
  REGION 1..6: note = Artificial sequence
VHGQER

SEQ ID NO: 69 - protein, 7 aa, synthetic construct
  REGION 1..7: note = Artificial sequence
PNTHGGP

SEQ ID NO: 70 - protein, 6 aa, synthetic construct
  REGION 1..6: note = Artificial sequence
VHGRER

SEQ ID NO: 71 - protein, 7 aa, synthetic construct
  REGION 1..7: note = Artificial sequence
SNFGNEK

SEQ ID NO: 72 - protein, 6 aa, synthetic construct
  REGION 1..6: note = Artificial sequence
VHGVER

SEQ ID NO: 73 - protein, 7 aa, synthetic construct
  REGION 1..7: note = Artificial sequence
TNIAGGS

SEQ ID NO: 74 - protein, 6 aa, synthetic construct
  REGION 1..6: note = Artificial sequence
VHGYAR

SEQ ID NO: 75 - protein, 6 aa, synthetic construct
  REGION 1..6: note = Artificial sequence
DRGSQS

SEQ ID NO: 76 - protein, 7 aa, synthetic construct
  REGION 1..7: note = Artificial sequence
YSNGDKE

SEQ ID NO: 77 - protein, 7 aa, synthetic construct
  REGION 1..7: note = Artificial sequence
SNFGNEK

SEQ ID NO: 78 - protein, 5 aa, synthetic construct
  REGION 1..5: note = Artificial sequence
SGHRS

SEQ ID NO: 79 - protein, 6 aa, synthetic construct
  REGION 1..6: note = Artificial sequence
FSETGR

SEQ ID NO: 80 - protein, 13 aa, synthetic construct
  REGION 1..13: note = Artificial sequence
CASSPWDSPNEQY

SEQ ID NO: 81 - protein, 6 aa, synthetic construct
  REGION 1..6: note = Artificial sequence
DRGSQS

SEQ ID NO: 82 - protein, 7 aa, synthetic construct
  REGION 1..7: note = Artificial sequence
YQAGDKE

SEQ ID NO: 83 - protein, 7 aa, synthetic construct
  REGION 1..7: note = Artificial sequence
GNDLGGI

SEQ ID NO: 84 - protein, 5 aa, synthetic construct
  REGION 1..5: note = Artificial sequence
SGHRS

SEQ ID NO: 85 - protein, 6 aa, synthetic construct
  REGION 1..6: note = Artificial sequence
VHGEER

SEQ ID NO: 86 - protein, 13 aa, synthetic construct
  REGION 1..13: note = Artificial sequence
CASSPWDSPNEQY

SEQ ID NO: 87 - protein, 6 aa, synthetic construct
  REGION 1..6: note = Artificial sequence
DRGSQS

SEQ ID NO: 88 - protein, 7 aa, synthetic construct
  REGION 1..7: note = Artificial sequence
YSNGDKE

SEQ ID NO: 89 - protein, 7 aa, synthetic construct
  REGION 1..7: note = Artificial sequence
DNPRGGM

SEQ ID NO: 90 - protein, 5 aa, synthetic construct
  REGION 1..5: note = Artificial sequence
QGHRA

SEQ ID NO: 91 - protein, 6 aa, synthetic construct
  REGION 1..6: note = Artificial sequence
VHGEER

SEQ ID NO: 92 - protein, 13 aa, synthetic construct
  REGION 1..13: note = Artificial sequence
CASSPWDSPNEQY

SEQ ID NO: 93 - protein, 6 aa, synthetic construct
  REGION 1..6: note = Artificial sequence
DRGSQS

SEQ ID NO: 94 - protein, 7 aa, synthetic construct
  REGION 1..7: note = Artificial sequence
YQAGDKE

SEQ ID NO: 95 - protein, 7 aa, synthetic construct
  REGION 1..7: note = Artificial sequence
GNAQGGM

SEQ ID NO: 96 - protein, 5 aa, synthetic construct
  REGION 1..5: note = Artificial sequence
PGHRA

SEQ ID NO: 97 - protein, 6 aa, synthetic construct
  REGION 1..6: note = Artificial sequence
VHGEER

SEQ ID NO: 98 - protein, 13 aa, synthetic construct
  REGION 1..13: note = Artificial sequence
CASSPWDSPNVQY

SEQ ID NO: 99 - protein, 6 aa, synthetic construct
  REGION 1..6: note = Artificial sequence
DRGSQS

SEQ ID NO: 100 - protein, 7 aa, synthetic construct
  REGION 1..7: note = Artificial sequence
YPQGDKK

SEQ ID NO: 101 - protein, 7 aa, synthetic construct
  REGION 1..7: note = Artificial sequence
DNPAGGI

SEQ ID NO: 102 - protein, 5 aa, synthetic construct
  REGION 1..5: note = Artificial sequence
SGHRS

SEQ ID NO: 103 - protein, 6 aa, synthetic construct
  REGION 1..6: note = Artificial sequence
VHGEER

SEQ ID NO: 104 - protein, 13 aa, synthetic construct
  REGION 1..13: note = Artificial sequence
CASSPWDSPNVQY

SEQ ID NO: 105 - protein, 6 aa, synthetic construct
  REGION 1..6: note = Artificial sequence
DRGSQS

SEQ ID NO: 106 - protein, 7 aa, synthetic construct
  REGION 1..7: note = Artificial sequence
YQEGDKE

SEQ ID NO: 107 - protein, 7 aa, synthetic construct
  REGION 1..7: note = Artificial sequence
SNFGNEK

SEQ ID NO: 108 - protein, 5 aa, synthetic construct
  REGION 1..5: note = Artificial sequence
PGHRA

SEQ ID NO: 109 - protein, 6 aa, synthetic construct
  REGION 1..6: note = Artificial sequence
VHGEER

SEQ ID NO: 110 - protein, 13 aa, synthetic construct
  REGION 1..13: note = Artificial sequence
CASSPWDSPNEQY

SEQ ID NO: 111 - protein, 6 aa, synthetic construct
  REGION 1..6: note = Artificial sequence
DRGSQS

SEQ ID NO: 112 - protein, 7 aa, synthetic construct
  REGION 1..7: note = Artificial sequence
YSQGDKE

SEQ ID NO: 113 - protein, 7 aa, synthetic construct
  REGION 1..7: note = Artificial sequence
DNPRGGM

SEQ ID NO: 114 - protein, 5 aa, synthetic construct
  REGION 1..5: note = Artificial sequence
PGHRS

SEQ ID NO: 115 - protein, 6 aa, synthetic construct
  REGION 1..6: note = Artificial sequence
VHGEER

SEQ ID NO: 116 - protein, 13 aa, synthetic construct
  REGION 1..13: note = Artificial sequence
CASSPWDSPNVQY

SEQ ID NO: 117 - protein, 6 aa, synthetic construct
  REGION 1..6: note = Artificial sequence
DRGSQS

SEQ ID NO: 118 - protein, 7 aa, synthetic construct
  REGION 1..7: note = Artificial sequence
YSNGDKE

SEQ ID NO: 119 - protein, 7 aa, synthetic construct
  REGION 1..7: note = Artificial sequence
DNEQGGM

SEQ ID NO: 120 - protein, 5 aa, synthetic construct
  REGION 1..5: note = Artificial sequence
PGHRS

SEQ ID NO: 121 - protein, 6 aa, synthetic construct
  REGION 1..6: note = Artificial sequence
VHGEER

SEQ ID NO: 122 - protein, 13 aa, synthetic construct
  REGION 1..13: note = Artificial sequence
CASSPWDSPNVQY

SEQ ID NO: 123 - protein, 6 aa, synthetic construct
  REGION 1..6: note = Artificial sequence
DRGSQS

SEQ ID NO: 124 - protein, 7 aa, synthetic construct
  REGION 1..7: note = Artificial sequence
YQEGDKE

SEQ ID NO: 125 - protein, 7 aa, synthetic construct
  REGION 1..7: note = Artificial sequence
NNPSGGM

SEQ ID NO: 126 - protein, 5 aa, synthetic construct
  REGION 1..5: note = Artificial sequence
QGHRA

SEQ ID NO: 127 - protein, 6 aa, synthetic construct
  REGION 1..6: note = Artificial sequence
VHGEER

SEQ ID NO: 128 - protein, 13 aa, synthetic construct
  REGION 1..13: note = Artificial sequence
CASSPWDSPNEQY

SEQ ID NO: 129 - protein, 6 aa, synthetic construct
  REGION 1..6: note = Artificial sequence
DRGSQS

SEQ ID NO: 130 - protein, 7 aa, synthetic construct
  REGION 1..7: note = Artificial sequence
YSQGDKE

SEQ ID NO: 131 - protein, 7 aa, synthetic construct
  REGION 1..7: note = Artificial sequence
DNPAGGI

SEQ ID NO: 132 - protein, 5 aa, synthetic construct
  REGION 1..5: note = Artificial sequence
SGHRS

SEQ ID NO: 133 - protein, 6 aa, synthetic construct
  REGION 1..6: note = Artificial sequence
VHGEER

SEQ ID NO: 134 - protein, 13 aa, synthetic construct
  REGION 1..13: note = Artificial sequence
CASSPWDSPNEQY

SEQ ID NO: 135 - protein, 6 aa, synthetic construct
  REGION 1..6: note = Artificial sequence
DRGSQS

SEQ ID NO: 136 - protein, 7 aa, synthetic construct
  REGION 1..7: note = Artificial sequence
YSQGDKE

SEQ ID NO: 137 - protein, 7 aa, synthetic construct
  REGION 1..7: note = Artificial sequence
NNPSGGM

SEQ ID NO: 138 - protein, 5 aa, synthetic construct
  REGION 1..5: note = Artificial sequence
PGHRS

SEQ ID NO: 139 - protein, 6 aa, synthetic construct
  REGION 1..6: note = Artificial sequence
VHGEER

SEQ ID NO: 140 - protein, 13 aa, synthetic construct
  REGION 1..13: note = Artificial sequence
CASSPWDSPNVQY

SEQ ID NO: 141 - protein, 232 aa, synthetic construct
  REGION 1..232: note = Artificial sequence
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK